(19) United States
(12) Patent Application Publication
(10) Pub. No.: US 2025/0002925 A1
(43) Pub. Date: Jan. 2, 2025

(54) COMPOSITIONS AND METHODS FOR ENHANCED PROTEIN PRODUCTION IN BACILLUS CELLS

(71) Applicant: DANISCO US INC., Palo Alto, CA (US)

(72) Inventors: Zhen Ma, Garnet Valley, PA (US); Ryan Frisch, Newark, DE (US); Brian Paul, Wilmington, PA (US); Steven Doig, Wilmington, DE (US); Stacey Robida-Stubbs, Woolwich Township, NJ (US)

(21) Appl. No.: 18/710,129

(22) PCT Filed: Nov. 11, 2022
(86) PCT No.: PCT/US2022/079687

§ 371 Date: May 14, 2024

Related U.S. Application Data
(60) Provisional application No. 63/279,813, filed on Nov. 16, 2021.

(51) Int. Cl. C12N 15/75; C12N 1/20; C12N 9/26; C12P 21/02; C12R 1/10

(52) U.S. Cl. CPC C12N 15/75; C12N 1/20; C12N 9/2414; C12P 21/02; C12R 2001/10; C12Y 302/01001

(57) ABSTRACT

Certain embodiments of the disclosure are related to recombinant Bacillus strains comprising enhanced protein productivity phenotypes, compositions and methods for constructing such recombinant Bacillus cells, and the like. The recombinant Bacillus strains described herein are particularly useful for the enhanced production of proteins of interest when grown/cultivated/fermented under suitable conditions.

### CROSS REFERENCE TO RELATED APPLICATIONS

This application claims benefit to U.S. Provisional Patent Application No. 63/279,813, filed Nov. 16, 2021, which is incorporated herein by referenced in its entirety.

### FIELD

The present disclosure is generally related to the fields of bacteriology, microbiology, genetics, molecular biology, enzymology, industrial protein production the like. Certain embodiments of the disclosure are related to recombinant Bacillus cells (strains) comprising enhanced protein productivity phenotypes, compositions and methods for constructing such recombinant (modified) Bacillus cells, and the like.

### REFERENCE TO A SEQUENCE LISTING

The contents of the electronic submission of the text file Sequence Listing, named “NB42009-WO-PCT_SequenceListing.xml” was created on Nov. 10, 2022 and is 103 KB in size, which is hereby incorporated by reference in its entirety.

### BACKGROUND

Gram-positive bacteria such as Bacillus subtilis, Bacillus licheniformis, Bacillus amyloliquefaciens and the like are frequently used as microbial factories for the production of industrial relevant proteins, due to their excellent fermentation properties and high yields (e.g., up to 25 grams per liter culture; Van Dijl and Hecker, 2013). For example, Bacillus sp. host cells are well known for their production of enzymes (e.g., amylases, cellulases, mannanases, pectate lysases, proteases, pullulanases, etc.) necessary for food, textile, laundry, medical instrument cleaning, pharmaceutical industries and the like. Because these non-pathogenic Gram-positive bacteria produce proteins that completely lack toxic by-products (e.g., lipopolysaccharides; LPS, also known as endotoxins) they have obtained the “Qualified Presumption of Safety” (QPS) status of the European Food Safety Authority (EFSA), and many of their products gained a “Generally Recognized As Safe” (GRAS) status from the US Food and Drug Administration (Olempska-Beer et al., 2006; Earl et al., 2008; Caspers et al., 2010).

Thus, the production of proteins (e.g., enzymes, antibodies, receptors, etc.) via microbial host cells is of particular interest in the biotechnological arts. Likewise, the optimization of Bacillus host cells for the production and secretion of one or more protein(s) of interest is of high relevance, particularly in the industrial biotechnology setting, wherein small improvements in protein yield are quite significant when the protein is produced in large industrial quantities. For example, the expression of many heterologous proteins can still be challenging and unpredictable with respect to yield and the like. As described hereinafter, the present disclosure is related to the highly desirable and unmet needs for obtaining and constructing Bacillus sp. cells (e.g., protein production hosts) having enhanced protein production capabilities.

### SUMMARY

As described hereinafter, certain aspects of the disclosure are related to compositions and methods for designing and constructing recombinant (modified) microbial host cells, such as recombinant Bacillus strains exemplified herein, which recombinant strains are particularly useful for the enhanced production of proteins of interest when cultivated under suitable conditions. Certain embodiments of the disclosure therefore provide, inter alia, one or more prsA gene expression cassettes suitable for introduction and integration at one or more defined B. licheniformis gene loci, prsA gene promoter sequences, prsA gene coding sequences (open reading frames), control cells, recombinant cells, proteins of interest, expression constructs (cassettes) encoding proteins of interest and the like.

In certain embodiments, the disclosure provides prsA gene expression cassettes. In certain aspects, prsA gene cassettes comprise an upstream (5′) prsA gene promoter sequence operably linked to a downstream (3′) prsA gene coding sequence (CDS). In related aspects, prsA gene cassettes may be referred to as 2^{nd} copy prsA gene cassettes. In other related aspects, the disclosure provides 2^{nd} copy prsA gene cassettes suitable for integration at a defined genomic locus of a desired host cell. In certain aspects, a prsA gene promoter sequence comprises at least 85% identity to SEQ ID NO: 29 and/or a prsA gene CDS comprises at least 80% identity to SEQ ID NO: 30.

Thus, certain other embodiments are related to recombinant B. licheniformis cells producing proteins of interest and comprising an introduced 2^{nd} copy prsA gene cassette integrated at defined locus. In certain aspects, recombinant B. licheniformis cells producing a protein of interest (POI) and having an introduced 2^{nd} copy prsA gene cassette integrated at a defined genomic locus (e.g., B. licheniformis amyL locus) produce increased amounts of the POI relative to control B. licheniformis cells producing the same POI, wherein the control cells comprise the same prsA gene cassette integrated at the catH locus, or wherein the control cells comprises a non-integrating copy of the same prsA gene cassette. In certain embodiments, a protein of interest (POI) is an enzyme.

Certain other embodiments provide methods for producing proteins of interest in B. licheniformis cells generally comprising constructing B. licheniformis cells producing a protein of interest (POI), introducing into the cells a 2^{nd} copy prsA gene cassette integrated at a defined genomic locus (e.g., B. licheniformis amyL locus) and fermenting the recombinant cells under suitable conditions for the production of the POI. Certain aspects are related to recombinant B. licheniformis cells producing an increased amount of the POI compared to control B. licheniformis cells producing the same POI, wherein the control cells comprise the same 2^{nd} copy prsA gene cassette integrated at the catH locus, or wherein the control cells comprise a non-integrating copy of the same prsA gene cassette. In certain embodiments of the methods, the prsA gene cassettes comprise a prsA gene promoter sequence comprising at least 85% identity to SEQ ID NO: 29 and/or comprise a prsA gene CDS comprising at least 80% identity to SEQ ID NO: 30. In certain other embodiments of the methods, a protein of interest (POI) is an enzyme.

### BRIEF DESCRIPTION OF THE BIOLOGICAL SEQUENCES

SEQ ID NO: 1 is a synthetic oligonucleotide (DNA) primer sequence 860.

SEQ ID NO: 2 is a synthetic oligonucleotide primer sequence 861.

SEQ ID NO: 3 is a synthetic oligonucleotide primer sequence 1636.

SEQ ID NO: 4 is a synthetic oligonucleotide primer sequence 1637.

SEQ ID NO: 5 is a synthetic oligonucleotide forward primer sequence.

SEQ ID NO: 6 is a synthetic oligonucleotide reverse primer sequence.

SEQ ID NO: 7 is a synthetic oligonucleotide forward primer sequence.

SEQ ID NO: 8 is a synthetic oligonucleotide reverse primer sequence.

SEQ ID NO: 9 is a synthetic oligonucleotide forward primer sequence.

SEQ ID NO: 10 is a synthetic oligonucleotide reverse primer sequence.

SEQ ID NO: 11 is a synthetic DNA editing template.

SEQ ID NO: 12 is a sequence verified plasmid isolate named “pRF1005”.

SEQ ID NO: 13 is the open reading frame (ORF) sequence of the B. licheniformis serA1 gene.

SEQ ID NO: 14 is the ORF sequence of the B. licheniformis lysA gene.

SEQ ID NO: 15 is the DNA sequence of the pB1.comK plasmid.

SEQ ID NO: 16 is the DNA sequence of the ΔrghR2 allele.

SEQ ID NO: 17 is a 1523 bp PCR product of the ΔrghR2 allele having a deletion of the rghR2 gene CDS, except for the first nine (9) and last nine (9) bp.

SEQ ID NO: 18 is a 1922 bp PCR product of the intact rghR2 allele

SEQ ID NO: 19 is the DNA sequence of the ΔdltA-2 allele.

SEQ ID NO: 20 is a 2067 bp PCR product of the ΔdltA-2 allele having a deletion of 700 bp of dltA-2 gene CDS.

SEQ ID NO: 21 is a 2767 bp PCR product of the intact dltA-2 allele.

SEQ ID NO: 22 is a DNA sequence of the linear PCR product targeting the amyL locus for integration of the introduced (2^{nd}) copy prsA cassette.

SEQ ID NO: 23 is the DNA sequence of the upstream (5′) homology arm for the amyL locus.

SEQ ID NO: 24 is the DNA sequence of the catH promoter.

SEQ ID NO: 25 is the DNA sequence encoding the CatH protein.

SEQ ID NO: 26 is the DNA sequence encoding a dual terminator sequence comprising of the catH terminator of SEQ ID NO: 27 operably linked to the spoVG terminator of SEQ ID NO: 28.

SEQ ID NO: 27 is the DNA sequence encoding the catH terminator.

SEQ ID NO: 28 is the DNA sequence encoding the spoVG terminator.

SEQ ID NO: 29 is the DNA sequence of the native B. licheniformis prsA promoter.

SEQ ID NO: 30 is an ORF sequence encoding the native B. licheniformis PrsA protein.

SEQ ID NO: 31 is the DNA sequence of the B. licheniformis amyL terminator.

SEQ ID NO: 32 is a downstream (3′) homology arm for the amyL locus.

SEQ ID NO: 33 is the DNA sequence of the introduced (2^{nd}) copy amyL integration cassette amyL::catH-prsAp-prsA.

SEQ ID NO: 34 is the 2698 bp sequence

SEQ ID NO: 35 is the 3562 bp sequence

SEQ ID NO: 36 is the DNA sequence encoding the reporter “amylase 1” (Amy1) protein.

SEQ ID NO: 37 is the DNA sequence of a synthetic p3 promoter.

SEQ ID NO: 38 is the DNA sequence of a B. subtilis modified aprE 5′-UTR.

SEQ ID NO: 39 is the DNA sequence encoding the B. licheniformis AmyL signal peptide.

SEQ ID NO: 40 is the DNA sequence of the B. licheniformis amyL transcriptional terminator.

SEQ ID NO: 41 is the DNA sequence of a synthetic p2 promoter.

SEQ ID NO: 42 is the DNA sequence encoding the reporter “amylase 2” (Amy2) protein.

SEQ ID NO: 43 is the amino acid sequence of the native B. licheniformis PrsA protein encoded by SEQ ID NO: 30.

### DETAILED DESCRIPTION

As described herein, certain embodiments of the disclosure are related to compositions and methods for enhanced protein production in Bacillus (host) cells. In particular, certain aspects of the disclosure provide recombinant Bacillus cells (strains) which are particularly useful for the enhanced production of proteins of interest when the recombinant cells are grown/cultivated/fermented under suitable conditions. More particularly, as set forth hereinafter, and further described in the Examples below, Applicant has surprisingly observed that recombinant B. licheniformis cells comprising an introduced 2^{nd} copy of a prsA gene expression cassette integrated at a defined B. licheniformis gene locus can produce increased amounts proteins of interest as compared to control B. licheniformis cells having the same introduced 2^{nd} copy of the prsA gene cassette integrated at the B. licheniformis catH locus. Thus, certain aspects of the disclosure provide, inter alia, one or more prsA gene expression cassettes suitable for introduction and integration at one or more pre-defined B. licheniformis gene loci, prsA gene promoter sequences, prsA gene coding sequences (open reading frames), control cells, recombinant cells, proteins of interest, expression constructs (cassettes) encoding proteins of interest and the like.

### I. Definitions

In view of the compositions and methods described herein, the following terms and phrases are defined. Terms not defined herein should be accorded their ordinary meaning as used in the art. Unless defined otherwise, all technical and scientific terms used herein have the same meaning as commonly understood by one of ordinary skill in the art to which the present compositions and methods apply. Although any methods and materials similar or equivalent to those described herein can also be used in the practice or testing of the present compositions and methods, representative illustrative methods and materials are now described.

All publications and patents cited herein are incorporated by reference in their entirety.

It is further noted that the claims may be drafted to exclude any optional element. As such, this statement is intended to serve as antecedent basis for use of such exclusive terminology as “solely,” “only”, “excluding”, “not including” and the like, in connection with the recitation of claim elements, or use of a “negative” limitation or proviso thereof. An example of a proviso includes phrases such as, “wherein the recombinant cell produces an increased amount of a protein of interest relative to the control cell producing the same protein of interest, wherein the control cell comprises a non-integrating 2^{nd} copy of the prsA gene cassette”.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, “the genus Bacillus” includes all species within the genus “Bacillus” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”.

As used herein, the terms “recombinant” or “non-natural” refer to an organism, microorganism, cell, nucleic acid molecule, or vector that has at least one engineered genetic alteration, or has been modified by the introduction of a heterologous nucleic acid molecule, or refer to a cell (e.g., a microbial cell) that has been altered such that the expression of a heterologous or endogenous nucleic acid molecule or gene can be controlled. Recombinant also refers to a cell that is derived from a non-natural cell or is progeny of a non-natural cell having one or more such modifications. Genetic alterations include, for example, modifications introducing expressible nucleic acid molecules encoding proteins, or other nucleic acid molecule additions, deletions, substitutions or other functional alteration of a cell's genetic material. For example, recombinant cells may express genes or other nucleic acid molecules that are not found in identical or homologous form within a native (wild-type) cell (e.g., a fusion or chimeric protein), or may provide an altered expression pattern of endogenous genes, such as being over-expressed, under-expressed, minimally expressed, or not expressed at all. “Recombination”, “recombining” or generating a “recombined” nucleic acid is generally the assembly of two or more nucleic acid fragments wherein the assembly gives rise to a chimeric gene.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein. It is understood that the polynucleotides (or nucleic acid molecules) described herein include “genes”, “vectors” and “plasmids”.

Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence (CDS).

As used herein, the term “coding sequence” (abbreviated “CDS”) refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

The term “promoter” as used herein refers to a nucleic acid sequence capable of controlling the expression of a coding sequence or functional RNA. In general, a coding sequence (CDS) is located 3′ (downstream) to a promoter sequence. Promoters may be derived in their entirety from a native gene (e.g., a prsA gene promoter), or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

As used herein, the plasmid named “pRF879” means the pRF879 plasmid of SEQ ID NO: 78 described in PCT Publication No. WO2021146411.

As used herein, the plasmid named “pZM221” means the pZM221 plasmid of SEQ ID NO: 84 described in PCT Publication No. WO2021146411.

As used herein, the plasmid named “pRF1005” (SEQ ID NO: 12) is a Cas9 plasmid targeting the catH locus, and comprises the editing template of SEQ ID NO: 11.

As used herein, a “wild-type (native) prsA gene” encodes a “wild-type (native) PrsA protein”.

As used herein, a wild-type (native) Bacillus licheniformis “prsA gene promoter” comprises the DNA sequence set forth in SEQ ID NO: 29.

As used herein, a wild-type B. licheniformis “prsA gene coding sequence (CDS)” comprises the open reading frame (ORF) set forth in SEQ ID NO: 30 and encodes a native PrsA protein. In certain aspects, functional PrsA proteins comprise a “protein chaperone” function or activity.

In certain embodiments, a wild-type prsA gene CDS comprises about 80% or greater (nucleotide) sequence identity to SEQ ID NO: 30. In other aspects, a wild-type prsA gene CDS comprises at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 30. In other embodiments, a wild-type prsA gene promoter comprises about 85% sequence identity to SEQ ID NO 29. In certain aspects, a wild-type prsA gene promoter comprises at least about 85%, 86%, 87%, 88%, 89%, 90%, 91%, 92%, 93%, 94%, 95%, 96%, 97%, 98%, 99% or 100% sequence identity to SEQ ID NO: 29.

As used herein, when a Bacillus host cell of the disclosure comprises an endogenous (native) “prsA gene” (i.e., encoding a native PrsA protein), and a polynucleotide (e.g., an integration expression cassette) encoding a native PrsA protein is introduced into the same Bacillus host cell, the introduced polynucleotide may be referred to herein as a “second (2^{nd}) copy prsA gene”.

As used herein, the phrase “2^{nd} copy prsA gene” means an introduced polynucleotide comprising a prsA gene CDS having at least 80% identity to the ORF of SEQ ID NO: 30. In certain aspects, the 2^{nd} copy prsA gene CDS can be expressed from a functional prsA gene promoter region comprising at least 85% identity to the prsA promoter region of SEQ ID NO: 29. In certain embodiments, an introduced 2^{nd} copy prsA gene comprises at least an upstream (5′) prsA gene promoter sequence operably linked to a downstream (3′) prsA gene CDS (e.g., 5′-[prsA gene promoter]-[prsA gene CDS]-3′).

As used herein, a B. licheniformis strain named “BF140” comprises deletions of the serA1 (ΔserA1; SEQ ID NO: 13) and the lysA genes (ΔlysA; SEQ ID NO: 14) and the introduced pB1.comK plasmid (SEQ ID NO: 15), as generally described in PCT Publication No. WO2019/40412.

As used herein, a B. licheniformis strain named “BF412” comprises deletions of serA1 (ΔserA1; SEQ ID NO: 13), lysA (ΔlysA; SEQ ID NO: 14) introduced plasmid pB1.comK (SEQ ID NO: 15) and a deleted rghR2 (ΔrghR2) allele, as generally described in PCT Publication No. WO2021/146411.

As used herein, a B. licheniformis strain named “BF772” comprises deletions of serA1 (ΔserA1; SEQ ID NO: 13), lysA (ΔlysA; SEQ ID NO: 14) introduced plasmid pB1.comK (SEQ ID NO: 15), a deleted rghR2 (ΔrghR2) allele, and a deleted dltA-2 (ΔdltA-2) allele, as generally described in WO2021/146411.

As used herein, a B. licheniformis isolate named “ZM1319” comprises deletions of serA1 (ΔserA1; SEQ ID NO: 13), lysA (ΔlysA; SEQ ID NO: 14) introduced plasmid pB1.comK (SEQ ID NO: 15), a deleted rghR2 (ΔrghR2) allele, a deleted dltA-2 (ΔdltA-2) allele and an introduced (2^{nd} copy) of prsA gene (e.g., 5′-[prsA promoter]-prsA gene coding sequence (ORF)]-3′) integrated at the amyZ locus.

As used herein, a B. licheniformis isolate named “ZM1322” comprises deletions of serA1 (ΔserA1; SEQ ID NO: 13), lysA (ΔlysA; SEQ ID NO: 14) introduced plasmid pB1.comK (SEQ ID NO: 15), a deleted rghR2 (ΔrghR2) allele, a deleted dltA-2 (ΔdltA-2) allele and an introduced (2^{nd} copy) of prsA gene (e.g., 5′-[prsA promoter]-prsA gene coding sequence (ORF)]-3′) integrated at the catH locus.

As used herein, a B. licheniformis isolate named “ZM1325” comprises deletions of serA1 (ΔserA1; SEQ ID NO: 13), lysA (ΔlysA; SEQ ID NO: 14) introduced plasmid pB1.comK (SEQ ID NO: 15), a deleted rghR2 (ΔrghR2) allele, a deleted dltA-2 (ΔdltA-2) allele and an introduced (2^{nd} copy) of prsA gene (e.g., 5′-[prsA promoter]-prsA gene coding sequence (ORF)]-3′) integrated at the amyL locus.

As used herein, a B. licheniformis isolate named “BF613” comprises deletions of serA1 (ΔserA1; SEQ ID NO: 13), lysA (ΔlysA; SEQ ID NO: 14) introduced plasmid pB1.comK (SEQ ID NO: 15), a deleted rghR2 (ΔrghR2) allele, a deleted dltA-2 (ΔdltA-2) allele and an introduced (2^{nd} copy) of prsA gene (e.g., 5′-[prsA promoter]-prsA gene coding sequence (ORF)]-3′) integrated at the catH locus.

As used herein, B. licheniformis strain “LDN665” is an amylase 1 (Amy1) reporter strain comprising 2 copies of Amy1 and 2^{nd} copy of prsA integrated at the catH locus.

As used herein, B. licheniformis strain “ZM1351” is an amylase 1 (Amy1) reporter strain comprising 2 copies of Amy1 and 2^{nd} copy of prsA integrated at the amyL locus.

As used herein, B. licheniformis strain “WAAA57” is an amylase 2 (Amy2) reporter strain comprising 2 copies of Amy2 and 2^{nd} copy of prsA integrated at the catH locus.

As used herein, B. licheniformis strain “WAAA197” is an amylase 2 (Amy2) reporter strain comprising 2 copies of Amy2 and 2^{nd} copy of prsA integrated at the amyL locus.

As used herein, the terms “Amylase 1” or “amylase 1” protein (abbreviated “Amy1”) refer to an amylase reporter protein, wherein the DNA encoding Amy1 reporter is set forth in SEQ ID NO: 36.

As used herein, the terms “Amylase 2” or “amylase 2” protein (abbreviated “Amy2”) refer to an amylase reporter protein, wherein the DNA encoding Amy2 reporter is set forth in SEQ ID NO: 42.

As used herein, a “host cell” refers to a cell that has the capacity to act as a host or expression vehicle for a newly introduced DNA sequence. Thus, in certain embodiments of the disclosure, the host cells are Gram-positive (e.g., Bacillus sp.) cells or Gram-negative E. coli cells.

As used herein, phrases such as “modified” cells and “daughter” cells refer to recombinant cells that comprise at least one genetic modification which is not present in the parent cells from which the modified cells were derived. In certain aspects, phrases such as “un-modified” cells, “parent” cells and/or “control” cells may be used when being compared with, or relative to, modified cells of the disclosure.

As used herein, when the expression of a protein of interest (POI) in a control cell is being compared to the expression of the same POI in a “modified” cell, it will be understood that the “control” and “modified” cells are grown/cultivated/fermented under the same conditions (e.g., the same conditions such as media, temperature, pH and the like). In certain embodiments, an increased amount of a protein of interest may be an endogenous Bacillus protein of interest (e.g., native proteases, native amylases, etc.), or a heterologous protein of interest (e.g., recombinant proteases, recombinant amylases, etc.) expressed in a recombinant Bacillus cell of the disclosure. In certain aspects, a POI is secreted into the culture media (broth).

As used herein, “increasing” protein production or “increased” protein production is meant an increased amount of protein produced (e.g., a protein of interest). The protein may be produced inside the host cell, or secreted (or transported) into the culture medium. In certain embodiments, the protein of interest is produced (secreted) into the culture medium. Increased protein production may be detected for example, as higher maximal level of protein or enzymatic activity (e.g., such as protease activity, amylase activity, pullulanase activity, cellulase activity, and the like), or total extracellular protein produced as compared to the parental cell.

As used herein, the terms “modification” and “genetic modification” are used interchangeably and include: (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) the down-regulation of a gene, (f) specific mutagenesis and/or (g) random mutagenesis of any one or more the genes disclosed herein.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any steps involved in the production of the polypeptide including, but not limited to, transcription, post-transcriptional modification, translation, post-translational modification, secretion and the like.

The term “operably linked” as used herein refers to the association of nucleic acid sequences on a single nucleic acid fragment so that the function of one is affected by the other. For example, a promoter is operably linked with a coding sequence (e.g., an ORF) when it is capable of affecting the expression of that coding sequence (i.e., that the coding sequence is under the transcriptional control of the promoter). Coding sequences can be operably linked to regulatory sequences in sense or antisense orientation.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “a functional promoter sequence controlling the expression of a gene of interest (or open reading frame thereof) linked to the gene of interest's protein coding sequence” refers to a promoter sequence which controls the transcription and translation of the coding sequence in Bacillus sp. cell. For example, in certain embodiments, the present disclosure is directed to a polynucleotide comprising a 5′ promoter (or 5′ promoter region, or tandem 5′ promoters and the like), wherein the promoter region is operably linked to a nucleic acid sequence (e.g., an ORF) encoding a protein.

As used herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure.

As used herein, the term “introducing”, as used in phrases such as “introducing into a bacterial cell” or “introducing into a Bacillus cell at least one polynucleotide open reading frame (ORF), or a gene thereof, or a vector thereof and the like, includes methods known in the art for introducing polynucleotides into a cell, including, but not limited to protoplast fusion, natural or artificial transformation (e.g., calcium chloride, electroporation), transduction, transfection, conjugation and the like.

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). Transformation therefore generally refers to introducing an exogenous DNA into a host cell so that the DNA is maintained as a chromosomal integrant or a self-replicating extra-chromosomal vector.

As used herein, “transforming DNA”, “transforming sequence”, and “DNA construct” refer to DNA that is used to introduce sequences into a host cell or organism. Transforming DNA is DNA used to introduce sequences into a host cell or organism. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein, “disruption of a gene” or a “gene disruption”, are used interchangeably and refer broadly to any genetic modification that substantially prevents a host cell from producing a functional gene product (e.g., a protein). Thus, as used herein, a gene disruption includes, but is not limited to, frameshift mutations, premature stop codons (i.e., such that a functional protein is not made), substitutions eliminating or reducing activity of the protein internal deletions (such that a functional protein is not made), insertions disrupting the coding sequence, mutations removing the operable link between a native promoter required for transcription and the open reading frame, and the like.

As used herein “an incoming sequence” refers to a DNA sequence that is introduced into the Bacillus sp. chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a nonfunctional gene or operon. In some embodiments, the non-functional sequence may be inserted into a gene to disrupt function of the gene. In another embodiment, the incoming sequence includes a selective marker. In a further embodiment the incoming sequence includes two homology boxes.

As used herein, “homology box” refers to a nucleic acid sequence, which is homologous to a sequence in the host cell chromosome. More specifically, a homology box is an upstream or downstream region having between about 80 and 100% sequence identity, between about 90 and 100% sequence identity, or between about 95 and 100% sequence identity with the immediate flanking coding region of a gene, or part of a gene to be deleted, disrupted, inactivated, down-regulated and the like, according to the invention. These sequences direct where in the chromosome a DNA construct is integrated and directs what part of the chromosome is replaced by the incoming sequence. While not meant to limit the present disclosure, a homology box may include about between 1 base pair (bp) to 200 kilobases (kb). In certain aspects, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, the term “selectable marker-encoding nucleotide sequence” refers to a nucleotide sequence which is capable of expression in the host cells and where expression of the selectable marker confers to cells containing the expressed gene the ability to grow in the presence of a corresponding selective agent or lack of an essential nutrient.

As used herein, the terms “selectable marker” and “selective marker” refer to a nucleic acid (e.g., a gene) capable of expression in host cell which allows for ease of selection of those hosts containing the vector. Examples of such selectable markers include, but are not limited to, antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

A “residing selectable marker” is one that is located on the chromosome of the microorganism to be transformed. A residing selectable marker encodes a gene that is different from the selectable marker on the transforming DNA construct. Selective markers are well known to those of skill in the art. As indicated above, the marker can be an antimicrobial resistance marker (e.g., amp^{R}, phleo^{R}, spec^{R}, kan^{R}, ery^{R}, tet^{R}, cmp^{R} and neo^{R}. In some embodiments, the present invention provides a chloramphenicol resistance gene (e.g., the gene present on pC194, as well as the resistance gene present in the Bacillus licheniformis genome). This resistance gene is particularly useful in the present invention, as well as in embodiments involving chromosomal amplification of chromosomally integrated cassettes and integrative plasmids. Other markers useful in accordance with the invention include, but are not limited to auxotrophic markers, such as serine (e.g., serA), lysine (e.g., lysA), tryptophan, and detection markers (e.g., β-galactosidase).

As used herein, a host cell “genome”, a bacterial (host) cell “genome”, or a Bacillus sp. (host) cell “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell. in some embodiments plasmids exist in a parental cell and are lost in the daughter cell.

A used herein, a “transformation cassette” refers to a specific vector comprising a gene (or ORF thereof), and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously or can integrate into a chromosome of a host organism).

An “expression vector” refers to a vector that has the ability to incorporate and express heterologous DNA in a cell. Many prokaryotic and eukaryotic expression vectors are commercially available and know to one skilled in the art. Selection of appropriate expression vectors is within the knowledge of one skilled in the art.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. For example, in certain embodiments, a parental B. licheniformis (host) cell is modified (e.g., transformed) by introducing therein one or more “targeting vectors”.

As used herein, the term “protein of interest” or “POI” refers to a protein of interest that is desired to be expressed in a recombinant host cell. In certain aspects, a modified host cell expresses the POI at increased levels relative to a control cell expressing the same POI. Thus, as used herein, a POI may be an enzyme, a substrate-binding protein, a surface-active protein, a structural protein, a receptor protein, and the like. In certain aspects, a modified cell of the disclosure produces an increased amount of a heterologous POI relative to the control cell. In particular embodiments, an increased amount of a POI produced by a modified cell is at least about 0.5% to 1.0% increased (or higher) relative to the control cell.

The phrase “gene of interest” (abbreviated, “GOI”) may be used herein when referring to a nucleic acid (gene, ORF, polynucleotide) encoding a protein of interest (POI). A “gene of interest (GOI)” encoding a “protein of interest (POI)” may be a naturally occurring gene, a mutated gene or a synthetic gene.

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In certain embodiments, a gene of the instant disclosure encodes a commercially relevant industrial protein of interest, such as an enzyme (e.g., a acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof).

As used herein, a “variant” polypeptide refers to a polypeptide that is derived from a parent (or reference) polypeptide by the substitution, addition, or deletion of one or more amino acids, typically by recombinant DNA techniques. Variant polypeptides may differ from a parent polypeptide by a small number of amino acid residues and may be defined by their level of primary amino acid sequence homology/identity with a parent (reference) polypeptide.

Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent (reference) polypeptide sequence. As used herein, a “variant” polynucleotide refers to a polynucleotide encoding a variant polypeptide, wherein the “variant polynucleotide” has a specified degree of sequence homology/identity with a parent polynucleotide, or hybridizes with a parent polynucleotide (or a complement thereof) under stringent hybridization conditions. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent (reference) polynucleotide sequence.

As used herein, a “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

As used herein, in the context of a polypeptide or a sequence thereof, the term “substitution” means the replacement (i.e., substitution) of one amino acid with another amino acid.

As defined herein, an “endogenous gene” refers to a gene in its natural location in the genome of an organism.

As defined herein, a “heterologous” gene, a “non-endogenous” gene, or a “foreign” gene refer to a gene (or ORF) not normally found in the host organism, but that is introduced into the host organism by gene transfer. As used herein, the term “foreign” gene(s) comprise native genes (or ORFs) inserted into a non-native organism and/or chimeric genes inserted into a native or non-native organism.

As defined herein, a “heterologous control sequence”, refers to a gene expression control sequence (e.g., a promoter or enhancer) which does not function in nature to regulate (control) the expression of the gene of interest. Generally, heterologous nucleic acid sequences are not endogenous (native) to the cell, or a part of the genome in which they are present, and have been added to the cell, by infection, transfection, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “derived” encompasses the terms “originated” “obtained,” “obtainable,” and “created,” and generally indicates that one specified material or composition finds its origin in another specified material or composition, or has features that can be described with reference to the another specified material or composition.

As used herein, the term “homology” relates to homologous polynucleotides or polypeptides.

If two or more polynucleotides or two or more polypeptides are homologous, this means that the homologous polynucleotides or polypeptides have a “degree of identity” of at least 60%, more preferably at least 70%, even more preferably at least 85%, still more preferably at least 90%, more preferably at least 95%, and most preferably at least 98%. Whether two polynucleotide or polypeptide sequences have a sufficiently high degree of identity to be homologous as defined herein, can suitably be investigated by aligning the two sequences using a computer program known in the art, such as “GAP” provided in the GCG program package (Program Manual for the Wisconsin Package, Version 8, August 1994, Genetics Computer Group, 575 Science Drive, Madison, Wisconsin, USA 53711) (Needleman and Wunsch, 1970). Using GAP with the following settings for DNA sequence comparison: GAP creation penalty of 5.0 and GAP extension penalty of 0.3.

As used herein, the term “percent (%) identity” refers to the level of nucleic acid or amino acid sequence identity between the nucleic acid sequences that encode a polypeptide or the polypeptide's amino acid sequences, when aligned using a sequence alignment program.

As used herein, “specific productivity” is total amount of protein produced per cell per time over a given time period.

As defined herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in preferred embodiments, it is on each side of the sequence being flanked. The sequence of each homology box is homologous to a sequence in the Bacillus chromosome. These sequences direct where in the Bacillus chromosome the new construct gets integrated and what part of the Bacillus chromosome will be replaced by the incoming sequence. In other embodiments, the 5′ and 3′ ends of a selective marker are flanked by a polynucleotide sequence comprising a section of the inactivating chromosomal segment. In some embodiments, a flanking sequence is present on only a single side (either 3′ or 5′), while in other embodiments, it is present on each side of the sequence being flanked.

### II. Expressing a Second Copy prsA Gene from Amy1 Locus Enhances Protein Production

The prsA gene of Bacillus subtilis, which encodes the PrsA protein, was initially defined by non-lethal mutations that decreased the secretion of several exoproteins (Kontinen and Sarvas, 1988). The PrsA protein has been described to act as a chaperone, and is translocated across the cytoplasmic membrane (Kontinen et al., 1993). PCT Publication No. WO1994/19471 describes a Gram-positive bacterial expression system, wherein an introduced copy of the B. subtilis prsA gene coding sequence (CDS) was overexpressed (overproduction) using various upstream promoters and sources, particularly specifying that overproduction of PrsA protein means an amount greater than wild-type. Likewise, US Patent Publication No. 2010/0255534 describes the overexpression of an introduced copy of the B. subtilis prsA gene CDS operably linked to a strong upstream promoter and comprising one or more deleted or inactivated genes selected from abrB, dltA, dltB, dltC, dltD and dltE. As concluded in these publications, overexpression (overproduction) of prsA (PrsA) in recombinant B. subtilis strains resulted in enhanced protein production vis-à-vis control B. subtilis strains that did not comprise the introduced prsA gene overexpression cassette. More recently, PCT Publication No. WO2021/146411 has described recombinant B. licheniformis strains comprising an introduced copy of the prsA gene (2^{nd} copy) integrated at the catH locus, wherein the introduced (2^{nd} copy) prsA gene comprised the native prsA gene promoter region operably linked to the native prsA gene CDS (e.g., 5′-[native prsA pro]-[native prsA ORF]-3′). As described in this publication, recombinant B. licheniformis strains comprising the 2^{nd} copy prsA cassette integrated at the catH locus produced increased amounts of reporter proteins compared to control B. licheniformis strains.

As generally set forth above, these contrasting results of modulated or variable prsA gene expression levels indicate that the choice of promoter and locus are not trivial. In particular, as shown below in TABLE 1, Applicant screened recombinant B. licheniformis strains expressing an amylase reporter protein (TABLE 1, second column) and comprising an introduced 2^{nd} copy prsA gene cassette (TABLE 1, first column).

TABLE 1
BACILLUS STRAINS EXPRESSING 2^{ND} COPY
PRSA GENE CASSETTE AND REPORTER PROTEIN

| 2^{nd} Copy prsA gene cassette | Rel. Amy Activity (+/−Std. Deviation) |
|---|---|
| 5′-[prsA pro]-[prsA CDS]-3′ | 1.00 ± 0.02 |
| 5′-[het pro_1]-[prsA CDS]-3′ | 0.87 ± 0.02 |
| 5′-[het pro_2]-[prsA CDS]-3′ | 0.95 ± 0.00 |
| 5′-[het pro_3]-[prsA CDS]-3′ | 0.93 ± 0.02 |
| 5′-[het pro_4]-[prsA CDS]-3′ | 0.71 ± 0.01 |

For example, as presented above in TABLE 1, protein productivity (amylase activity) of recombinant B. licheniformis strains expressing a 2^{nd} copy prsA gene CDS under the control of a heterologous promoter (e.g., heterologous promoter (pro) 1-4; abbreviated, “5′-[het pro_1]”, etc.) were compared to protein productivity of the recombinant strain expressing the 2^{nd} copy prsA gene CDS under the control of its native promoter (i.e., 5′-[prsA pro]). As revealed in TABLE 1, protein productivity was reduced in strains expressing a 2^{nd} copy prsA gene CDS under the control of a heterologous promoter when compared to productivity of strains expressing a 2^{nd} copy prsA gene CDS under the control of its native prsA promoter, further demonstrating that the choice/selection of a promoter region for driving expression of a prsA gene CDS is not a trivial or readily predictable endeavor.

More particularly, as described hereinafter and set forth below in the Examples, Applicant has surprisingly observed that recombinant B. licheniformis cells comprising an introduced 2^{nd} copy of a prsA expression cassette integrated at an optimized and defined B. licheniformis gene locus can produce increased amounts of reporter proteins as compared to control B. licheniformis cells comprising the same introduced 2^{nd} copy of the prsA expression cassette integrated at the B. licheniformis catH locus. Thus, certain aspects of the disclosure are related to prsA integration (expression) constructs specifically designed to target and integrate at one or more B. licheniformis genomic loci described and contemplated herein.

More specifically, Examples 1 and 2 set forth below, generally describe the design and construction of plasmids suitable for targeted prsA gene integration (2^{nd} copy) at defined B. licheniformis genomic loci, the design and construction of recombinant B. licheniformis strains suitable for testing and screening the 2^{nd} copy prsA integration cassettes, and the like. In particular, as generally described in Examples 1 and 2, the B. licheniformis cells comprising an introduced 2^{nd} copy of the prsA (integration) cassettes were constructed, wherein the prsA cassettes were integrated at the catH locus or amyL locus.

As described in Example 3, expression cassettes encoding a reporter protein (Amy1) were introduced into B. licheniformis strain BF613 (with prsA cassette integrated at the catH locus) and B. licheniformis strain ZM1325 (with prsA cassette integrated at the amyL locus). For example, as shown in TABLE 6 (Example 3), the two (2) Amy1 production strains (LDN665 and ZM1351) were assayed for production of Amy1 using standard small scale conditions, demonstrating an improvement of Amy1 production in strains with the 2^{nd} copy prsA cassette integrated at the amyL locus (ZM1351) relative to strains comprising the 2^{nd} copy prsA cassette integrated at the catH locus (LDN665).

As further described in Example 4, expression cassettes encoding a second amylase reporter protein (Amy2) were introduced into B. licheniformis strains BF613 and ZM1325. For example, as shown in TABLE 7 (Example 4), the two (2) Amy2 production strains (WAAA57 and WAAA197) were assayed for production of Amy2 using standard small scale conditions, demonstrating an improvement of Amy2 production in the strains comprising the 2^{nd} copy prsA cassette integrated at the amyL locus (WAAA197) relative to strains comprising the 2^{nd} copy prsA cassette integrated at the of catH locus (WAAA57).

Thus, certain aspects of the disclosure are related to prsA gene coding sequences (CDS) comprising sequence homology to a prsA gene CDS described herein, and/or prsA gene promoter sequences comprising homology to a prsA gene promoter sequence described herein. In certain related aspects, a prsA gene CDS encodes an active (functional) PrsA protein. For example, in certain embodiments, a prsA gene CDS encodes PrsA protein comprising at least about 50% identity to the mature PrsA amino acid sequence of SEQ ID NO: 43. In certain aspects, a PrsA protein comprising at least about 50% identity to the PrsA protein of SEQ ID NO: 43 is further defined as a functional or active PrsA protein.

### III. Recombinant Polynucleotides and Molecular Biology

As generally described above and hereinafter, certain aspects of the disclosure are related to recombinant Bacillus strains producing proteins of interest. More particularly, as presented below in the Examples, recombinant polynucleotides (vectors, expression cassettes, etc.), recombinant (modified) Bacillus strains and the like are readily constructed using routine molecular biology and microbiology techniques and methods know to one skilled in the art. Therefore, the instant disclosure generally relies on routine techniques in the field of recombinant genetics. Basic texts disclosing the general methods of use in present disclosure include Sambrook et al., (2^{nd} Edition, 1989); Kriegler (1990) and Ausubel et al., (1994). Likewise, those of skill in the art are well aware of suitable methods for introducing polynucleotide sequences into bacterial cells (e.g., E. coli, Bacilli, etc.).

Thus, in certain embodiments, the disclosure provides, inter alia, recombinant Bacillus cells comprising an introduced 2^{nd} copy of a prsA expression cassette integrated at a defined gene locus, compositions and methods for design and construction of recombinant Bacillus cells producing proteins of interest and comprising an introduced 2^{nd} copy of a prsA expression cassette integrated at a defined gene locus, compositions and methods for producing increased amounts of proteins of interest, and the like.

In certain embodiments, a prsA gene coding sequence (CDS) comprises homology to the DNA sequence of SEQ ID NO: 30. For example, in certain aspects, a prsA gene CDS comprises at least about 50% identity to the prsA gene CDS of SEQ ID NO: 30. In certain aspects, a prsA gene CDS comprises at least 55%, at least 60%, at least 65%, at least 70%, at least 75%, at least 80%, at least 85%, at least 90%, at least 95% and up to 100% identity to the prsA gene CDS of SEQ ID NO: 30. In certain embodiments, a prsA gene CDS comprises 80%, 81%, 82%, 83%, 84%, 85%, 86%, 87%, 88%, 89%, 90%, 91%, 92%, 93%, 94%, 95%, 96%, 97%, 98%, 98%, 99% or 100% identity to the prsA gene CDS of SEQ ID NO: 30.

In certain other aspects, a prsA gene promoter sequence comprises homology to the DNA sequence of SEQ ID NO: 29. For example, in certain aspects, a prsA gene promoter comprises at least about 50% identity to the prsA gene promoter of SEQ ID NO: 29. In certain aspects, a prsA gene promoter comprises at least 55%, at least 60%, at least 65%, at least 70%, at least 75%, at least 80%, at least 85%, at least 90%, at least 95% and up to 100% identity to the prsA gene promoter of SEQ ID NO: 29. In certain embodiments, a prsA gene promoter comprises 85%, 86%, 87%, 88%, 89%, 90%, 91%, 92%, 93%, 94%, 95%, 96%, 97%, 98%, 98%, 99% or 100% identity to the prsA gene promoter of SEQ ID NO: 29.

Certain aspects of the disclosure are therefore related to recombinant polynucleotides (e.g., plasmids, vectors, DNA constructs, etc.), recombinant host cells, expression cassettes encoding a 2^{nd} copy of a prsA gene CDS, compositions and methods for constructing recombinant polynucleotides, recombinant Bacillus host cells, and the like.

Thus, in certain embodiments, a polynucleotide (genes, vectors, plasmids, DNA elements, etc.) of the disclosure may be genetically modified, wherein genetic modifications include, but are not limited to, (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) the down-regulation of a gene (e.g., interfering RNA), (f) specific mutagenesis and/or (g) random mutagenesis of any one or more the genes disclosed herein. In certain embodiments, a modified Bacillus cell of the disclosure is constructed by increasing the expression of a gene and/or by reducing (or eliminating) the expression of a gene, using methods well known in the art, for example, insertions, disruptions, replacements, or deletions. The portion of the gene to be modified or inactivated may be, for example, the coding region (CDS, ORF) or a regulatory (DNA) element required for expression of the coding region. An example of such a regulatory or control sequences may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator sequence, a transcriptional activator sequence and the like.

Gene deletion techniques enable the partial or complete removal of gene(s), thereby eliminating their expression, or expressing a non-functional (or reduced activity) protein product. In such methods, the deletion of the gene(s) may be accomplished by homologous recombination using a plasmid that has been constructed to contiguously contain the 5′ and 3′ regions flanking the gene. The contiguous 5′ and 3′ regions may be introduced into a Bacillus cell, for example, on a temperature-sensitive plasmid, such as pE194, in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is effected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers. Thus, a person of skill in the art may readily identify nucleotide regions in the gene's coding sequence and/or the gene's non-coding sequence suitable for complete or partial deletion. In other embodiments, a modified Bacillus cell of the disclosure is constructed by introducing, substituting, or removing one or more nucleotides in the gene or a regulatory element required for the transcription or translation thereof.

In certain embodiments, a modified Bacillus cell is constructed via CRISPR-Cas9 editing. For example, a wild-type gene encoding a native protein of interest (or functional variant protein of interest thereof) may be modified via CRISPR-Cas9 editing, by means of nucleic acid guided endonucleases, that find their target DNA by binding either a guide RNA (e.g., Cas9) and Cpf1 or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template (e.g., an editing template to replace the native gene promoter sequence with a heterologous promoter). For example, the gene encoding the nucleic acid guided endonuclease (for this purpose Cas9 from S. pyogenes) or a codon optimized gene encoding the Cas9 nuclease is operably linked to a promoter active in the Bacillus cell and a terminator active in Bacillus cell, thereby creating a Bacillus Cas9 expression cassette. Likewise, one or more target sites unique to the gene of interest are readily identified by a person skilled in the art. For example, to build a DNA construct encoding a gRNA-directed to a target site within the gene of interest using Streptococcus pyogenes Cas9, the variable targeting domain (VT) will comprise nucleotides of the target site which are 5′ of the (PAM) proto-spacer adjacent motif (NGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a Bacillus expression cassette for the gRNA is created by operably linking the DNA encoding the gRNA to a promoter active in Bacillus cells and a terminator active in Bacillus cells.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500-bp 5′ of targeted gene can be fused to about 500-bp 3′ of the targeted gene to generate an editing template, which template is used by the Bacillus host's machinery to repair the DNA break generated by the RGEN. The Cas9 expression cassette, the gRNA expression cassette and the editing template can be co-delivered to the cells using many different methods. The transformed cells are screened by PCR amplifying the target gene locus, by amplifying the locus with a forward and reverse primer. These primers can amplify the wild-type locus or the modified locus that has been edited by the RGEN. These fragments are then sequenced using a sequencing primer to identify edited colonies.

In other embodiments, a modified Bacillus cell is constructed by random or specific mutagenesis using methods well known in the art, including, but not limited to, chemical mutagenesis and transposition. Modification of the gene may be performed by subjecting the parental cell to mutagenesis and screening for mutant cells in which expression of the gene has been altered. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods. Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions, and selecting for mutant cells exhibiting reduced or no expression of the gene.

International PCT Publication No. WO2003/083125 discloses methods for modifying Bacillus cells, such as the creation of Bacillus deletion strains and DNA constructs using PCR fusion to bypass E. coli. PCT Publication No. WO2002/14490 discloses methods for modifying Bacillus cells including (1) the construction and transformation of an integrative plasmid (pComK), (2) random mutagenesis of coding sequences, signal sequences and pro-peptide sequences, (3) homologous recombination, (4) increasing transformation efficiency by adding non-homologous flanks to the transformation DNA, (5) optimizing double cross-over integrations, (6) site directed mutagenesis and (7) marker-less deletion. Those of skill in the art are well aware of suitable methods for introducing polynucleotide sequences into bacterial cells (e.g., E. coli and Bacillus). Indeed, such methods as transformation including protoplast transformation and congression, transduction, and protoplast fusion are known and suited for use in the present disclosure. Methods of transformation are particularly preferred to introduce a DNA construct of the present disclosure into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the modified Bacillus strain by methods known in the art. In some embodiments, resolution of the vector from a host chromosome leaves the flanking regions in the chromosome, while removing the indigenous chromosomal region.

Promoters and promoter sequence regions for use in the expression of genes, open reading frames (ORFs) thereof and/or variant sequences thereof in Bacillus cells are generally known on one of skill in the art. Promoter sequences of the disclosure are generally chosen so that they are functional in the Bacillus cells, and include, but are not limited to, naturally occurring promoter sequences, synthetic promoter sequences, and/or promoter sequence combinations thereof and the like, which promoter (sequences) are operable/functional in Bacillus cells. Examples of synthetic (engineered) promoters capable of producing heterologous (foreign) proteins in Bacillus cells include, but are not limited to, the promoter systems described by Zhou et al. (2019), Wang et al. (2019) and Castillo-Hair et al. (2019). Certain other exemplary Bacillus promoter sequences include, but are not limited to, the B. subtilis alkaline protease (aprE) promoter, the α-amylase promoter of B. subtilis, the α-amylase promoter of B. amyloliquefaciens, the neutral protease (nprE) promoter from B. subtilis, a mutant aprE promoter (e.g., PCT Publication No. WO2001/51643), a B licheniformis tuf promoter, a B licheniformis citZ promoter, or any other functional promoter from Bacillus sp. cells. In certain embodiments, a heterologous promoter is used to drive the expression of a protein of interest or a prsA gene CDS. Methods for screening and creating promoter libraries with a range of activities (promoter strength) in Bacillus cells is describe in PCT Publication No. WO2003/089604.

### IV. Fermenting Bacillus Cells for Production of a Protein of Interest

In certain embodiments, the disclosure provides recombinant microbial cells of producing proteins of interest. More particularly, certain aspects are related genetically modified (recombinant) microbial cells expressing heterologous polynucleotides encoding proteins of interest. Thus, particular embodiments are related to growing, cultivating, fermenting and the like, microbial cells for the production of proteins of interest. In general, fermentation methods well known in the art are used to ferment the microbial cells.

In some embodiments, the cells are grown under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within batch cultures, cells progress through a static lag phase to a high growth log phase and finally to a stationary phase, where growth rate is diminished or halted. If untreated, cells in the stationary phase eventually die. In general, cells in log phase are responsible for the bulk of production of product.

A suitable variation on the standard batch system is the “fed-batch fermentation” system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO2. Batch and fed-batch fermentations are common and well known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Culturing/fermenting is generally accomplished in a growth medium comprising an aqueous mineral salts medium, organic growth factors, a carbon and energy source material, molecular oxygen, and, of course, a starting inoculum of the microbial host to be employed.

In addition to the carbon and energy source, oxygen, assimilable nitrogen, and an inoculum of the microorganism, it is necessary to supply suitable amounts in proper proportions of mineral nutrients to assure proper microorganism growth, maximize the assimilation of the carbon and energy source by the cells in the microbial conversion process, and achieve maximum cellular yields with maximum cell density in the fermentation media.

The composition of the aqueous mineral medium can vary over a wide range, depending in part on the microorganism and substrate employed, as is known in the art. The mineral media should include, in addition to nitrogen, suitable amounts of phosphorus, magnesium, calcium, potassium, sulfur, and sodium, in suitable soluble assimilable ionic and combined forms, and also present preferably should be certain trace elements such as copper, manganese, molybdenum, zinc, iron, boron, and iodine, and others, again in suitable soluble assimilable form, all as known in the art.

The fermentation reaction is an aerobic process in which the molecular oxygen needed is supplied by a molecular oxygen-containing gas such as air, oxygen-enriched air, or even substantially pure molecular oxygen, provided to maintain the contents of the fermentation vessel with a suitable oxygen partial pressure effective in assisting the microorganism species to grow in a thriving fashion.

The fermentation temperature can vary somewhat, but for most microbial cells the temperature generally will be within the range of about 20° C. to 40° C.

The microorganisms also require a source of assimilable nitrogen. The source of assimilable nitrogen can be any nitrogen-containing compound or compounds capable of releasing nitrogen in a form suitable for metabolic utilization by the microorganism. While a variety of organic nitrogen source compounds, such as protein hydrolysates, can be employed, usually cheap nitrogen-containing compounds such as ammonia, ammonium hydroxide, urea, and various ammonium salts such as ammonium phosphate, ammonium sulfate, ammonium pyrophosphate, ammonium chloride, or various other ammonium compounds can be utilized. Ammonia gas itself is convenient for large scale operations, and can be employed by bubbling through the aqueous ferment (fermentation medium) in suitable amounts. At the same time, such ammonia can also be employed to assist in pH control.

The pH range in the aqueous microbial ferment (fermentation admixture) should be in the exemplary range of about 2.0 to 8.0. Preferences for pH range of microorganisms are dependent on the media employed to some extent, as well as the particular microorganism, and thus change somewhat with change in media as can be readily determined by those skilled in the art.

Preferably, the fermentation is conducted in such a manner that the carbon-containing substrate can be controlled as a limiting factor, thereby providing good conversion of the carbon-containing substrate to cells and avoiding contamination of the cells with a substantial amount of unconverted substrate. The latter is not a problem with water-soluble substrates, since any remaining traces are readily washed off. It may be a problem, however, in the case of non-water-soluble substrates, and require added product-treatment steps such as suitable washing steps.

As described above, the time to reach this level is not critical and may vary with the particular microorganism and fermentation process being conducted. However, it is well known in the art how to determine the carbon source concentration in the fermentation medium and whether or not the desired level of carbon source has been achieved.

If desired, part or all of the carbon and energy source material and/or part of the assimilable nitrogen source such as ammonia can be added to the aqueous mineral medium prior to feeding the aqueous mineral medium to the fermenter.

Each of the streams introduced into the reactor preferably is controlled at a predetermined rate, or in response to a need determinable by monitoring such as concentration of the carbon and energy substrate, pH, dissolved oxygen, oxygen or carbon dioxide in the off-gases from the fermenter, cell density measurable by dry cell weights, light transmittancy, or the like. The feed rates of the various materials can be varied so as to obtain as rapid a cell growth rate as possible, consistent with efficient utilization of the carbon and energy source, to obtain as high a yield of microorganism cells relative to substrate charge as possible.

In either a batch, or the preferred fed batch operation, all equipment, reactor, or fermentation means, vessel or container, piping, attendant circulating or cooling devices, and the like, are initially sterilized, usually by employing steam such as at about 121° C. for at least about 15 minutes. The sterilized reactor then is inoculated with a culture of the selected microorganism in the presence of all the required nutrients, including oxygen, and the carbon-containing substrate. The type of fermenter employed is not critical.

### V. Proteins of Interest

A protein of interest (POI) can be any endogenous or heterologous protein, and it may be a variant of such a POI. The protein can contain one or more disulfide bridges, or is a protein whose functional form is a monomer or a multimer (i.e., the protein has a quaternary (4°) structure and is composed of a plurality of identical (homologous) or non-identical (heterologous) subunits). In certain aspects, recombinant cells of the disclosure express/produce one or more endogenous proteins of interest, one or more heterologous proteins of interest, combinations thereof and the like.

In certain embodiments, a modified cell may produce an increased amount of a POI relative to a parental (or control) cell, wherein the increased amount of the POI is at least about a 0.01% increase, at least about a 0.10% increase, at least about a 0.50% increase, at least about a 1.0% increase, at least about a 2.0% increase, at least about a 3.0% increase, at least about a 4.0% increase, at least about a 5.0% increase, or an increase greater than 5.0%. In certain aspects, an increased amount of a POI is determined by assaying enzymatic activity, assaying protein function, assaying/quantifying specific productivity (Qp) and the like. For example, one skilled in the art may utilize routine methods and techniques known in the art for detecting, assaying, measuring, etc. protein expression, production, secretion and the like.

In certain embodiments, a POI or a variant POI thereof is an enzyme. In certain aspects, the enzyme is selected from the group consisting of acetyl esterases, aminopeptidases, amylases, arabinases, arabinofuranosidases, arylesterases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, lysozymes, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phosphodiesterases, phytases, polyesterases, polygalacturonases, proteases, peptidases, rhamno-galacturonases, ribonucleases, transferases, transport proteins, transglutaminases, xylanases, hexose oxidases, and combinations thereof.

In related aspects, a POI or a variant POI thereof is an enzyme selected from an Enzyme Commission (EC) Number: EC 1, EC 2, EC 3, EC 4, EC 5 or EC 6. For example, in certain embodiments a POI is an oxidoreductase enzyme. In other embodiments a POI is a transferase enzyme. In other embodiments a POI is a hydrolase enzyme. In other embodiments a POI is a lyase enzyme. In certain other embodiments a POI is an isomerase enzyme. In yet other embodiments, a POI is a ligase enzyme.

In certain aspects, an enzyme is a protease (e.g., a neutral protease, metalloproteases) and alkaline (or “serine”) proteases. For example, Bacillus subtilisin proteins (enzymes) are exemplary serine proteases for use in the present disclosure. A wide variety of Bacillus subtilisins have been identified and sequenced, for example, subtilisin 168, subtilisin BPN′, subtilisin Carlsberg, subtilisin DY, subtilisin 147 and subtilisin 309. In some embodiments of the present disclosure, the modified Bacillus cells produce mutant (i.e., variant) proteases. Thus, in certain embodiments, modified Bacillus cells comprise an expression construct encoding a protease. In certain other embodiments, modified Bacillus cells comprise an expression construct encoding an amylase. A wide variety of amylase enzymes and variants thereof are known to one skilled in the art.

In other embodiments, a POI or variant POI expressed and produced in a modified cell is a peptide, a peptide hormone, a growth factor, a clotting factor, a chemokine, a cytokine, a lymphokine, an antibody, a receptor, an adhesion molecule, a microbial antigen (e.g., HBV surface antigen, HPV E7, etc.), variants thereof, fragments thereof and the like. Other types of proteins (or variants thereof) of interest may be those that are capable of providing nutritional value to a food or to a crop. Non-limiting examples include plant proteins that can inhibit the formation of anti-nutritive factors and plant proteins that have a more desirable amino acid composition (e.g., a higher lysine content than a non-transgenic plant).

As briefly stated above, there are various assays known to those of skill in the art for detecting and measuring activity of intracellularly and extracellularly expressed proteins. In particular, for proteases, there are assays based on the release of acid-soluble peptides from casein or hemoglobin measured as absorbance at 280 nm or colorimetrically, using the Folin method. Other exemplary assays include succinyl-Ala-Ala-Pro-Phe-para-nitroanilide assay (SAAPFpNA) and the 2,4,6-trinitrobenzene sulfonate sodium salt assay (TNBS assay). International PCT Publication No. WO2014/164777 discloses Ceralpha α-amylase activity assays useful for amylase activities described herein. Means for determining the levels of secretion of a protein of interest in a host cell and detecting expressed proteins include the use of immunoassays with either polyclonal or monoclonal antibodies specific for the protein. Examples include enzyme-linked immunosorbent assay (ELISA), radioimmunoassay (RIA), fluorescence immunoassay (FIA), and fluorescent activated cell sorting (FACS).

### VI. Exemplary Embodiments

Non-limiting embodiments of compositions and methods disclosed herein are as follows:
1. A prsA gene cassette integrated at the amyL locus of a Bacillus licheniformis cell.
2. The cassette of embodiment 1, comprising an upstream (5′) prsA gene promoter sequence operably linked to a downstream (3′) prsA gene coding sequence (CDS).
3. The cassette of embodiment 2, wherein the prsA gene promoter sequence comprises at least 85% identity to SEQ ID NO: 29
4. The cassette of embodiment 2, wherein the prsA gene CDS comprises at least 80% identity to SEQ ID NO: 30.
5. A recombinant Bacillus licheniformis cell producing a protein of interest (POI) and comprising a prsA gene cassette integrated at the amyL locus.
6. The recombinant cell of embodiment 5, producing an increased amount of the POI relative to a control Bacillus cell producing the same POI, wherein the control cell comprises the same prsA gene cassette integrated at the catH locus.
7. The recombinant cell of embodiment 5, producing an increased amount of the POI relative to a control Bacillus cell producing the same POI, wherein the control cell comprises a non-integrating prsA gene cassette.
8. The recombinant cell of embodiment 5, wherein cassette comprises a prsA gene coding sequence (CDS) comprising at least 80% identity to SEQ ID NO: 30.
9. The recombinant cell of embodiment 5, wherein cassette comprises a promoter sequence comprising at least 85% identity to SEQ ID NO: 29.
10. The recombinant cell of embodiment 5, wherein the POI is an enzyme.
11. A method for producing a protein of interest (POI) in a Bacillus licheniformis cell comprising: obtaining or constructing a Bacillus cell producing a POI, introducing into the cell a prsA gene cassette integrated at the amyL locus, and fermenting the recombinant cell under suitable conditions for the production of the POI.
12. The method of embodiment 11, wherein the recombinant cell produces an increased amount of the POI relative to a control B. licheniformis cell producing the same POI, wherein the control cell comprises the same prsA gene cassette integrated at the catH locus.
13. The method of embodiment 11, wherein the recombinant cell produces an increased amount of the POI relative to a control B. licheniformis cell producing the same POI, wherein the control cell comprises a non-integrating prsA gene cassette.
14. The method of embodiment 11, wherein the cassette comprises a prsA gene coding sequence (CDS) comprising at least 80% identity to SEQ ID NO: 30.
15. The method of embodiment 11, wherein the cassette comprises a promoter sequence comprising at least 85% identity to SEQ ID NO: 29.
16. The method of embodiment 11, wherein the POI is an enzyme.

### EXAMPLES

Certain aspects of the present invention may be further understood in light of the following examples, which should not be construed as limiting. Modifications to materials and methods will be apparent to those skilled in the art. Standard recombinant DNA and molecular cloning techniques used herein are well known in the art (Ausubel et al., 1987; Sambrook et al., 1989). As described herein, all expression cassettes were transformed into the host strains using the methods described PCT Publication No. WO2019/040412 (incorporated herein by referenced in its entirety).

### Example 1

Construction of Plasmid pRF1005 Targeting catH Locus

In the present example, a Cas9 plasmid pRF1005 targeting the catH locus was constructed and comprises the editing template (SEQ ID NO: 11). The plasmid backbone was amplified by PCR from pRF946 described in PCT Publication No. WO2021/146411 (incorporated herein by reference in its entirety) using primers 860 (SEQ ID NO: 1; TABLE 2) and 861 (SEQ ID NO: 2; TABLE 2), and the editing template insert was amplified by PCR from a synthetic template using primers 1636 (SEQ ID NO: 3; TABLE 2) and 1637 (SEQ ID NO: 4; TABLE 2). The two parts were assembled using NEBuilder according to manufacturer's instructions and transformed into E. coli. A sequence verified isolate was stored as pRF1005 (SEQ ID NO: 12). Plasmid pRF1005 was used to delete the catH gene at the amyL locus. Rolling-circle amplification (RCA) was used to amplify the plasmid and make the plasmids suitable substrates for transformation using the TempliPhi amplification kit (GE Healthcare).

TABLE 2
FORWARD AND REVERSE PRIMERS
FOR PLASMID pRF1005

| SEQ ID | Primer | Sequence |
|---|---|---|
| 1 | 860 | TGTCAGACCAAGTTTACTCA |
| 2 | 861 | GAATTCCTCCATTTTCTTCTG |
| 3 | 1636 | TGAGTAAACTTGGTCTGACA |
| 4 | 1637 | CAGAAGAAAATGGAGGAATTC |

### Example 2

### Construction of Modified Host Strains

Recombinant B licheniformis strains described herein may be constructed by one of skill using any suitable B licheniformis host (e.g., see PCT Publication Nos. WO2019/40412 and WO2021/146411; each incorporated herein by reference in its entirety). For example, in certain aspects, host modifications can be introduced into a B. licheniformis strain such as BF140 (ΔserA1_ΔlysA), comprising deletions of serA1 and lysA, as generally described in PCT Publication No. WO2019/40412. In other aspects, host modifications can be introduced into a B. licheniformis strain (e.g., BF140) further comprising one or more genetic modifications including, but not limited to a modified dltA gene and/or a modified rghR2 gene, as generally described in PCT Publication No. WO2021/146411.

In the present example, a series of host modifications were introduced into a parental B. licheniformis strain BF140 comprising deletions of the serA1 (SEQ ID NO: 13) and the lysA genes (SEQ ID NO: 14), and containing the pB1.comK plasmid (SEQ ID NO: 15), as described in WO2019/40412. More specifically, a deleted rghR2 (ΔrghR2) allele was first constructed in the BF140 strain as described in WO2021/146411. Briefly, a version of BF140 containing the pB1.comK plasmid (SEQ ID NO:15) was made competent (WO2021/146411). One hundred (100) μl of competent cells were mixed with five (5) μl of pRF879 (SEQ ID NO:78; WO2021/146411) RCA and incubated at 1400 RPM and 37° C. for one and a half (1.5) hours. The mixtures were plated on L agar plates containing twenty (20) ppm kanamycin to select for cells transformed with the plasmid. The colonies were screened for the ΔrghR2 allele (SEQ ID NO: 16), a deletion of the rghR2 coding sequence except for the first nine (9) and last nine (9) bp, using standard PCR techniques and the primers in TABLE 3 below. Colonies with the ΔrghR2 allele (SEQ ID NO: 16) produce a PCR product of 1523 bp (SEQ ID NO: 17) using the forward (SEQ ID NO: 5) and reverse (SEQ ID NO: 6) primers set forth below in TABLE 3, while the parental cells containing the intact rghR2 gene produce a PCR product of 1922 bp (SEQ ID NO: 18). A colony containing the ΔrghR2 allele was stored as BF412.

TABLE 3
FORWARD AND REVERSE PRIMER PAIR

| SEQ ID | Primer | Sequence |
|---|---|---|
| 5 | Forward | GGATACGCCGATTTCAATGGC |
| 6 | Reverse | GGCTATGTGCTGGGGGAATT |

A version of BF412 containing the pB1.comK plasmid (SEQ ID NO: 15) was made competent (WO2021/146411). One hundred (100) μl of competent cells were mixed with five (5) μl of pZM221 (SEQ ID NO: 84; WO2021146411) RCA and incubated at 1400 RPM and 37° C. for one and a half (1.5) hours. The mixtures were plated on the L agar plates containing twenty (20) ppm kanamycin. The colonies were screened for the ΔdltA-2 allele (SEQ ID NO: 19), a deletion of 700 bp of the dltA coding sequence using standard PCR techniques, and the forward (SEQ ID NO: 7) and reverse (SEQ ID NO: 8) primers set forth below in TABLE 4. Colonies with the ΔdltA-2 allele produce a PCR product of 2067 bp (SEQ ID NO: 20) with the primers in TABLE 4, while the parental cells containing the intact dltA gene produce a PCR product of 2767 bp (SEQ ID NO: 21). This can be differentiated using standard electrophoresis techniques. A colony containing the 700 bp internal deletion of dltA (SEQ ID NO: 19) was stored as BF772.

TABLE 4
FORWARD AND REVERSE PRIMER PAIR

| SEQ ID | Primer | Sequence |
|---|---|---|
| 7 | Forward | GGGTACCTCCATGGTAAAGT |
| 8 | Reverse | ACGTATTAATGCAGTAGCCG |

A version of strain BF772 containing the pB1.comK plasmid (SEQ ID NO: 15) was made competent (WO2021/146411) and was transformed with a linear PCR product targeting the amyL locus for integration of the introduced (2^{nd} copy) of the prsA gene (SEQ ID NO: 22). For example, the targeting construct (cassette) comprises an upstream (5′) homology arm to the amyL locus (SEQ ID NO: 23) operably linked to the catH promoter (SEQ ID NO: 24) operably linked to the DNA encoding the CatH protein (SEQ ID NO: 25) operably linked to a dual terminator (SEQ ID NO: 26) composed of the catH terminator (SEQ ID NO: 27) operably linked to the spoVG terminator of B. subtilis (SEQ ID NO: 28). The construct further comprises the B. licheniformis prsA promoter (SEQ ID NO: 29) operably linked to the B. licheniformis prsA CDS (SEQ ID NO: 30) operably linked to the terminator from the amyL gene of B. licheniformis (SEQ ID NO: 31) operably linked to a downstream homology arm for the amyL locus (SEQ ID NO: 32).

Colonies that formed on L agar containing ten (10) ppm chloramphenicol were screened using colony PCR to confirm the modification of the amyL locus using standard PCR techniques with the forward (SEQ ID NO: 9) and reverse (SEQ ID NO: 10) primers set forth below in TABLE 5. Colonies containing the cassette (SEQ ID NO: 33) integrated at the amyL locus produced a PCR product of 3562 bp. The PCR product (SEQ ID NO: 35) was sequenced using the method of Sanger and an isolate was stored as ZM1319.

TABLE 5
FORWARD AND REVERSE PRIMER PAIR

| SEQ ID | Primer | Sequence |
|---|---|---|
| 9 | Forward | CGCTTCTTGAAAACGAGGTG |
| 10 | Reverse | GCTCATCCAAATCGATCCCA |

A version of ZM1319 containing the pB1.comK plasmid (SEQ ID NO: 15) was made competent (WO2021/146411). One hundred (100) μl of competent cells were mixed with five (5) μl of pRF1005 RCA and incubated at 1400 RPM and 37° C. for one and a half (1.5) hours. The mixtures were plated on the L agar plates containing twenty (20) ppm kanamycin. The colonies were screened for the deletion of the DNA encoding the 3′ end of the catH promoter (SEQ ID NO: 24) and the DNA sequence encoding the CatH protein (SEQ ID NO: 25), while retaining the amyL::prsAp-prsA cassette (SEQ ID NO: 33) using standard PCR techniques, and the forward (SEQ ID NO: 9) and reverse (SEQ ID NO: 10) primers set forth above in TABLE 5.

Correct colonies containing the (2^{nd} copy) prsA cassette integrated at the amyL locus (amyL::prsAp-prsA cassette; SEQ ID NO: 33) produced a PCR of 2698 bp (SEQ ID NO: 34), as opposed to the parent colonies containing (2^{nd} copy) prsA cassette integrated at the catH locus (amyL::catH-prsAp-prsA cassette; SEQ ID NO: 22), which produced a PCR product of 3562 bp in length (SEQ ID NO: 35). The difference was assessed visually using standard gel electrophoresis techniques and by sequencing the PCR product using the method of Sanger. An isolate was stored as ZM1322.

A version of ZM1322, named ZM1325, containing pB1.comK plasmid (SEQ ID NO: 15), was used in the following examples as host strains for amylase reporter production, wherein the ZM1325 strain contains the same host modifications as compared to the previously described host strain BF613 (WO2021/146411), except that the introduced (2^{nd} copy) prsA cassette was integrated at the amyL locus in the ZM1325 strain, while the same introduced (2^{nd} copy) prsA cassette was integrated at the catH locus in the BF613 strain.

### Example 3

Enhanced Amylase 1 Production in Bacillus Cells Comprising a Second Copy prsA Gene Integrated at the Amy1 Locus

In the present example, expression cassettes encoding a variant α-amylase (herein, “amylase 1”, abbreviated “Amy1”) were introduced into B. licheniformis strains BF613 and ZM1325 (i.e., comprising deletions of serA1 and lysA genes). More particularly, BF613 and ZM1325 are isogenic strains, except that strain BF613 contains the introduced (2^{nd} copy) prsA expression cassette integrated at the catH locus, while ZM1325 contains the same introduced (2^{nd} copy) prsA expression cassette integrated at the amyL locus.

For example, a first (1st) cassette of amylase 1 (Amy1; SEQ ID NO: 36) was integrated into the serA1 locus and contains the synthetic p3 promoter (SEQ ID NO: 37) operably linked to the modified B. subtilis aprE 5′-UTR (SEQ ID NO: 38) operably linked to the DNA encoding B. licheniformis AmyL signal peptide sequence (SEQ ID NO: 39) operably linked to the DNA encoding Amy1 (SEQ ID NO: 36) operably linked to the B. licheniformis amyL transcriptional terminator (SEQ ID NO: 40) operably linked to the serA1 ORF (SEQ ID NO: 13). A second (2″x) cassette of Amy1 (SEQ ID NO: 36) was integrated into the lysA locus and contains the lysA ORF (SEQ ID NO: 14) and the B. licheniformis p2 promoter (SEQ ID NO: 41) operably linked to the modified B. subtilis aprE 5′-UTR (SEQ ID NO: 38) operably linked to the DNA encoding B. licheniformis AmyL signal peptide sequence (SEQ ID NO:39) operably linked to the DNA encoding Amy1 (SEQ ID NO: 36) operably linked to the B. licheniformis amyL transcriptional terminator (SEQ ID NO: 40). This resulted in the Amy1 production strains named LDN665 (i.e., strain BF613 with 2 copies of Amy1 at lysA and serA loci) and ZM1351 (i.e., strain ZM1325 with 2 copies of Amy1 at lysA and serA loci).

The two Amy1 production strains (LDN665 and ZM1351) were assayed for production of α-amylase using standard small scale conditions (as described in PCT publication No. WO2018/156705 and WO2019/055261, each incorporated herein by reference). For example, the amylase reporter protein (Amy1) produced was quantified using the method of Bradford or the Ceralpha assay, wherein the assay results are shown in TABLE 6, demonstrating an improvement of Amy1 production in the strains comprising the introduced (2^{nd} copy) of the prsA gene integrated at the amyL locus (ZM1351) instead of catH locus (LDN665).

TABLE 6
RELATIVE EXPRESSION OF AMY1 IN STRAINS
COMPRISING 2^{ND} COPY prsA GENE INTEGRATED
AT DIFFERENT GENOMIC LOCI

| Strain | Description | Relative Amy1 Production |
|---|---|---|
| LDN665 | 2 copies Amy1; catH::prsA | 1.00 |
| ZM1351 | 2 copies Amy1; amyL::prsA | 1.07 |

### Example 4

Enhanced Amylase 2 Production in Bacillus Cells Comprising a Second Copy prsA Gene Integrated at the amyL Locus

In the instant example, expression cassettes encoding a different amylase reporter protein [Triple-A 21508-1] (herein, “amylase 2”, abbreviated “Amy2”) were introduced into B. licheniformis strain BF613 and ZM1325 (i.e., comprising deletions of serA1 and lysA genes). As set forth above, BF613 and ZM1325 are isogenic strains, except that strain BF613 contains the introduced (2^{nd} copy) prsA expression cassette integrated at the catH locus, while ZM1325 contains the same introduced (2^{nd} copy) prsA expression cassette integrated at the amyL locus. For example, a first (1st) cassette of Amy2 (SEQ ID NO: 42) was integrated into the serA1 locus and contains the serA1 ORF (SEQ ID NO: 13) operably linked to the synthetic p3 promoter (SEQ ID NO: 37) operably linked to the modified B. subtilis aprE 5′-UTR (SEQ ID NO: 38) operably linked to the DNA encoding B. licheniformis AmyL signal peptide sequence (SEQ ID NO: 39) operably linked to the DNA encoding Amy2 (SEQ ID NO: 42) operably linked to the B. licheniformis amyL transcriptional terminator (SEQ ID NO: 40). A second (2^{nd}) cassette of Amy2 was integrated into the lysA locus and contains the lysA ORF (SEQ ID NO: 14) and the B. licheniformis p2 promoter (SEQ ID NO: 41) operably linked to the modified B. subtilis aprE 5′-UTR (SEQ ID NO: 38) operably linked to the DNA encoding B. licheniformis AmyL signal peptide sequence (SEQ ID NO: 39) operably linked to the DNA encoding Amy2 (SEQ ID NO: 42) operably linked to the B. licheniformis amyL transcriptional terminator (SEQ ID NO: 40). This resulted in the Amy2 production strains named WAAA57 (i.e., strain BF613 with 2 copies of Amy2 at lysA and serA loci), and WAAA197 (i.e., strain ZM1325 with 2 copies of Amy1 at lysA and serA loci).

The two Amy2 production strains (WAAA57 and WAAA197) were assayed for production of amylase (using standard small scale conditions as described in Example 3), wherein the reporter assay results are shown in TABLE 7, demonstrating an improvement of Amy2 production in the strains comprising the introduced (2^{nd} copy) of the prsA gene integrated at the amyL locus (WAAA197), instead of catH locus (WAAA57).

TABLE 7
RELATIVE EXPRESSION OF AMY2 IN STRAINS
COMPRISING 2^{ND} COPY prsA GENE INTEGRATED
AT DIFFERENT GENOMIC LOCI

| Strain | Description | Relative Amy2 Production |
|---|---|---|
| WAAA57 | 2 copies Amy2; catH::prsA | 1.00 |
| WAAA197 | 2 copies Amy2; amyL::prsA | 1.04 |

### REFERENCES

PCT Publication No. WO1994/19471
PCT Publication No. WO2002/14490
PCT Publication No. WO2003/083125
PCT Publication No. WO2003/089604
PCT Publication No. WO2019/40412
PCT Publication No. WO2021/146411
PCT Publication No. WO2014/164777
US Patent Publication No. US20100255534
Caspers et al., “Improvement of Sec-dependent secretion of a heterologous model protein in Bacillus subtilis by saturation mutagenesis of the N-domain of the AmyE signal peptide”, Appl. Microbiol. Biotechnol., 86 (6): 1877-1885, 2010.
Castillo-Hair et al., “An Engineered B. subtilis Inducible Promoter System with over 10 000-Fold Dynamic Range”, ACS Synth. Biol., 8 (7): 1673-1678, 2019.
Earl et al., “Ecology and genomics of Bacillus subtilis”, Trends in Microbiology., 16 (6): 269-275, 2008.
Kontinen and Sarvas, “Mutants of Bacillus subtilis Defective in Protein Export”, J. Gen. Microbiology, 134, pages 2333-2344, 1988.
Kontinen et al., “A gene (prsA) of Bacillus subtilis involved in a novel, late stage of protein export”, Mol. Microbiology, 5, pages 1273-1283, 1991.
Olempska-Beer et al., “Food-processing enzymes from recombinant microorganisms—a review” Regul. Toxicol. Pharmacol., 45 (2): 144-158, 2006.
Van Dijl and Hecker, “Bacillus subtilis: from soil bacterium to super-secreting cell factory”, Microbial Cell Factories, 12 (3). 2013.
Wang et al., “Engineering strong and stress-responsive promoters in Bacillus subtilis by interlocking sigma factor binding motifs”, Synth. Syst. Biotechnol., 4 (4): 197-203, 2019.
Zhou et al., “Promoter engineering enables overproduction of foreign proteins from a single copy expression cassette in Bacillus subtilis”, Microbial Cell Factories, 18 (111), 2019.

1. A prsA gene cassette integrated at the amyL locus of a Bacillus licheniformis cell, wherein the cassette comprises an upstream prsA promoter sequence operably linked to a downstream prsA coding sequence (CDS).

2. (canceled)

3. The cassette of claim 1, wherein the prsA promoter sequence comprises at least 85% identity to SEQ ID NO: 29.

4. The cassette of claim 1, wherein the prsA CDS comprises at least 80% identity to SEQ ID NO: 30.

5. A recombinant Bacillus licheniformis cell producing a protein of interest (POI) and comprising an introduced a prsA gene cassette integrated at the amyL locus, wherein the cassette comprises an upstream prsA promoter sequence operably linked to a downstream prsA coding sequence (CDS).

6. The recombinant cell of claim 5, producing an increased amount of the POI relative to a control Bacillus cell producing the same POI, wherein the control cell comprises the same prsA gene cassette integrated at the catH locus.

7. The recombinant cell of claim 5, producing an increased amount of the POI relative to a control Bacillus cell producing the same POI, wherein the control cell comprises an introduced non-integrating copy of the same prsA gene cassette.

8. The recombinant cell of claim 5, wherein the prsA CDS comprises at least 80% identity to SEQ ID NO: 30.

9. The recombinant cell of claim 5, wherein the prsA promoter sequence comprises at least 85% identity to SEQ ID NO: 29.

10. The recombinant cell of claim 5, wherein the POI is an enzyme.

11. A method for producing a protein of interest (POI) in a Bacillus licheniformis cell comprising obtaining or constructing a B. licheniformis cell producing a POI, introducing into the cell a prsA gene cassette integrated at the amyL locus, and fermenting the recombinant cell under suitable conditions for the production of the POI, wherein the prsA gene cassette comprises an upstream prsA promoter sequence operably linked to a downstream prsA coding sequence (CDS).

12. The method of claim 11, wherein the recombinant cell produces an increased amount of the POI relative to a control B. licheniformis cell producing the same POI, wherein the control cell comprises the same prsA gene cassette integrated at the catH locus.

13. The method of claim 11, wherein the recombinant cell produces an increased amount of the POI relative to a control B. licheniformis cell producing the same POI, wherein the control cell comprises an introduced non-integrating copy of the same prsA gene cassette.

14. The method of claim 11, wherein the prsA CDS comprises at least 80% identity to SEQ ID NO: 30.

15. The method of claim 11, wherein the prsA promoter sequence comprises at least 85% identity to SEQ ID NO: 29.

16. The method of claim 11, wherein the POI is an enzyme.


SEQUENCE LISTING (43 sequences)

SEQ ID NO: 1 - DNA, 20 bp, synthetic construct
  misc_feature 1..20: note = synthetic
  source 1..20: mol_type = other DNA
tgtcagaccaagtttactca

SEQ ID NO: 2 - DNA, 21 bp, synthetic construct
  misc_feature 1..21: note = synthetic
  source 1..21: mol_type = other DNA
gaattcctccattttcttctg

SEQ ID NO: 3 - DNA, 20 bp, synthetic construct
  misc_feature 1..20: note = synthetic
  source 1..20: mol_type = other DNA
tgagtaaacttggtctgaca

SEQ ID NO: 4 - DNA, 21 bp, synthetic construct
  misc_feature 1..21: note = synthetic
  source 1..21: mol_type = other DNA
cagaagaaaatggaggaattc

SEQ ID NO: 5 - DNA, 21 bp, synthetic construct
  misc_feature 1..21: note = synthetic
  source 1..21: mol_type = other DNA
ggatacgccgatttcaatggc

SEQ ID NO: 6 - DNA, 20 bp, synthetic construct
  misc_feature 1..20: note = synthetic
  source 1..20: mol_type = other DNA
ggctatgtgctgggggaatt

SEQ ID NO: 7 - DNA, 20 bp, synthetic construct
  misc_feature 1..20: note = synthetic
  source 1..20: mol_type = other DNA
gggtacctccatggtaaagt

SEQ ID NO: 8 - DNA, 20 bp, synthetic construct
  misc_feature 1..20: note = synthetic
  source 1..20: mol_type = other DNA
acgtattaatgcagtagccg

SEQ ID NO: 9 - DNA, 20 bp, synthetic construct
  misc_feature 1..20: note = synthetic
  source 1..20: mol_type = other DNA
cgcttcttgaaaacgaggtg

SEQ ID NO: 10 - DNA, 20 bp, synthetic construct
  misc_feature 1..20: note = synthetic
  source 1..20: mol_type = other DNA
gctcatccaaatcgatccca

SEQ ID NO: 11 - DNA, 1000 bp, synthetic construct
  misc_feature 1..1000: note = synthetic
  source 1..1000: mol_type = other DNA
tgtgcttggaagcagagcccaatattatcccgaaacgataaaacggatgctgaaggaaggaaacgaagtcggcaaccattcctgggaccatccgttattgacaaggctgtcaaacgaaaaagcgtatcaggagattaacgacacgcaagaaatgatcgaaaaaatcagcggacacctgcctgtacacttgcgtcctccatacggcgggatcaatgattccgtccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgtccgcagatgctgctgaagagattattaaaaagctgaaagcaaaaggctatcaattggtaactgtatctcagcttgaagaagtgaagaagcagagaggctattgaataaatgagtagaaagcgccatatcggctgacagaggaaaggccgatatgattcggccttttttatatgtacttcttagcgggtctctaaaataaccaaaaagcaaggactgctgaaagggctgacataagccttttgccggcggtccttttttaattctgatttttcaaacttagccccctcgagaaaaccgcttgagtatcctccgctcatgttattgacctccttttcttgatcaatatatttgtatgagcggaaagaaaatttgactagacggatgcccctctgttgaaaatctttttacagcatgtcagaatatgatatgatacaattcaaaggaaagtttaaactgttatgattaggagtgtttgcatttatgaagaagattgcaattgcggcgattacagcgacaagcgtgctggctctcagcgcatgcagcgggggagattctgaggttgttgcggaaacaaaagctggaaatattacaaaagaagacctttatcaaacattaaaagacaatgccggagc

SEQ ID NO: 12 - DNA, 9724 bp, synthetic construct
  misc_feature 1..9724: note = synthetic
  source 1..9724: mol_type = other DNA
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggagagcttctttttcttgagcagttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatgtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgtgcttggaagcagagcccaatattatcccgaaacgataaaacggatgctgaaggaaggaaacgaagtcggcaaccattcctgggaccatccgttattgacaaggctgtcaaacgaaaaagcgtatcaggagattaacgacacgcaagaaatgatcgaaaaaatcagcggacacctgcctgtacacttgcgtcctccatacggcgggatcaatgattccgtccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgtccgcagatgctgctgaagagattattaaaaagctgaaagcaaaaggctatcaattggtaactgtatctcagcttgaagaagtgaagaagcagagaggctattgaataaatgagtagaaagcgccatatcggctgacagaggaaaggccgatatgattcggccttttttatatgtacttcttagcgggtctctaaaataaccaaaaagcaaggactgctgaaagggctgacataagccttttgccggcggtccttttttaattctgatttttcaaacttagccccctcgagaaaaccgcttgagtatcctccgctcatgttattgacctccttttcttgatcaatatatttgtatgagcggaaagaaaatttgactagacggatgcccctctgttgaaaatctttttacagcatgtcagaatatgatatgatacaattcaaaggaaagtttaaactgttatgattaggagtgtttgcatttatgaagaagattgcaattgcggcgattacagcgacaagcgtgctggctctcagcgcatgcagcgggggagattctgaggttgttgcggaaacaaaagctggaaatattacaaaagaagacctttatcaaacattaaaagacaatgccggagcgaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatccatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 13 - DNA, 1578 bp, Bacillus licheniformis
  source 1..1578: mol_type = other DNA
atgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaa

SEQ ID NO: 14 - DNA, 1320 bp, Bacillus licheniformis
  source 1..1320: mol_type = other DNA
ttgtttttacacggtactagcagacaaaatgaaagagggcacctcgaaatcggcggtgtcgatgttctatcattggcagaaagatacggaacacctctttatgtatacgatgtcgcgctgattagagagcgcgcccgaaaattccagaaggcattcaaggaagccggtttaaaagcgcaggtagcgtatgcaagcaaggcgttttcatcggttgccatgattcagcttgccgaacaagaggggctgtctctggatgtggtatcgggaggagagcttttcactgcgatcaaagcagggttcccagctgagcggattcattttcacggaaacaataagagccctgaagaactagccatggcgctggagcatcaaatcggctgcatcgtgctcgataactttcacgagatcgccattacagaagatctttgcaagcgatcaggacaaactgtagacgttttgctcagaatcactccgggagttgaagcgcacacgcacgattatattacgacggggcaggaagattccaaattcggttttgatctgcataatggacaggtcgaacaagccatcgaacaagtcctccgctcgtctgcgtttaagctcctcggcgtgcactgccacatcggttcgcaaatttttgatacggcaggatttgtccttgcagcagacaagattttcgagaagcttgcggaatggcgggagacttactctttcattccggaagtgctcaatcttggcgggggcttcggcatccgctatacaaaagacgacgagccgcttgcagctgatgtttatgttgaaaaaatcatcgaggcggtcaaagcaaatgccgagcatttcggctttgacatccctgagatttggatcgaaccaggccggtctctcgtcggtgatgcggggactacgctgtacacgatcggttctcaaaaagaggtgccgggcattcgcaaatatgtagccatcgacggcggcatgagcgataatatcaggccggcgctttatgaggcaaaatatgaagcagccgtcgccaacaggatgaacgatgcttgtcatgataccgcatcaatcgcaggaaaatgctgcgaaagcggagatatgctgatttgggatttggaaatccccgaagttcgcgacggagatgtgctcgccgttttctgcaccggtgcgtacggctacagcatggccaacaactacaaccgcattccgcgcccggccgtcgtctttgtcgaggacggggaagcgcagctcgtcattcagagagagacgtatgaggatatcgtcaagctggatctgccgctgaaatcgaaagtcaaacaataa

SEQ ID NO: 15 - DNA, 6393 bp, synthetic construct
  misc_feature 1..6393: note = synthetic
  source 1..6393: mol_type = other DNA
aagcttcatatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatggatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgggcgcgccggtatccgcgcttcttgagcactatttattcaaagccgctccagatcaatagcgctttttcagctccctgaggatgaattcgtatatcagctgattccggtcttctttcggatagagcataaattcctgtttcttctgcatggggtttccttcaatcctgtcgataaattttgttctcagccatgccgttcggtaaacctggttttcgaaagatgagatggatacgggcagctccagcgtttccccgttgacaaacgtgacaaacgtgttgtcatactttgccgcgcaaaactcgtgaacatgcgcatgggaaagccacccgcactgaggacgagttgaggaaaatgtggggaaaagaaaaatgttgtttgagtgatccaccatgatcggcggtttatgggaaactttaatgacttcatatgtgcccgcttttcttcccgcatagctcgatccgaaatagcggcagcttctttcgataatttgaaacggcttcatattgacgcggaaagtcctgtcggtctcaagtatttttgaggcggatttctccccctcacccagaggcaggacagccattgtcgaactgtttacttcatacgtatcctttgtcatatcctctgtgctcatgtgatttcccccttaaaaataaattcattcaaatacagatgcattttatttcatatagtaagtacatcacctattagtttgttgtttaaacaaactaacttattttcatcttatataacctcgtcagtattttcaatattttttttagttttttatgaacacattagatttaataaagggaagattcgctatgtactatgttgatacttaatttaaagattaaacaaatggagtggatgaagtggatatcgctgatcaaacctttgtcaaaaaagtaaatcaaaagttattattaaaagaaatccttaaaaattcacctatttcaagagcaaaattatctgaaatgactggattaaataaatcaactgtctcatcacaggtaaacacgttaatgaaagaaagtatggtatttgaaataggtcaaggacaatcaagtggcggaagaagacctgtcatgcttgtttttaataaaaaggcaggatactccgttggaatagatgttggtgtggattatattaatggcattttaacagaccttgaaggaacaatcgttcttgatcaataccgccatttggaatccaattctccagaaataacgaaagacattttgattgatatgattcatcactttattacgcaaatgccccaatctccgtacgggtttattggtataggtacttgcgtgcctggactcattgataaagatcaaaaaattgttttcactccgaactccaactggagagatattgacttaaaatcttcgatacaagagaagtacaatgtgtctgtttttattgaaaatgaggcaaatgctggcgcatatggagaaaaactatttggagctgcaaaaaatcacgataacattatttacgtaagtatcagcacaggaatagggatcggtgttattatcaacaatcatttatatagaggagtaagcggcttctctggagaaatgggacatatgacaatagactttaatggtcctaaatgcagttgcggaaaccgaggatgctgggaattgtatgcttcagagaaggctttatttaaatctcttcagaccaaagagaaaaaactgtcctatcaagatatcataaacctcgcccatctgaatgatatcggaaccttaaatgcattacaaaattttggattctatttaggaataggccttaccaatattctaaatactctcaacccacaagccgtaattttaagaaatagcataattgaatcgcatcctatggttttaaattcaatgagaagtgaagtatcatcaagggtttattcccaattaggcaatagctatgaattattgccatcttccttaggacagaatgcaccggcattaggaatgtcctccattgtgattgatcattttctggacatgattacaatgtaattttttatggaatggacagctcatctttaaagatgagtttttttattctaggagtatttctgaagcaatagtgacatggcaccttctcatatgaaaaaggagttctaaaataaaaatctcctttttcatgtgcaaattatttttctttataacgaaaatatctaaatgacaatgcatatgcaagaggggatcacataaatatatattttaaaaatatcccactttatccaattttcgtttgttgaactaatgggtgctttagttgaagaataaaagaccacattaaaaaatgtggtcttttgtgtttttttaaaggatttgagcgtagcgaaaaatccttttctttcttatcttgatactatatagaaacaacatcatttttcaaaattaggtcaaagccttgtgtatcaagggtttgatggttctttgacaggtaaaaactccttctgctattattaaatactatatagaaacaacatcatttttcaaaattaggtcaaagccttgtgtatcaagggtttgatggttctttgacaggtaaaaactccttctgctattattaaggtgtcgaatcaaaataatagaatgctagagaactagctcagaaggagtttttttgttgatttattcatctgaaaatgattatagcatcctcgaagataaaaccgcaacaggtaaaaagcgggattggaaggggaaaaagagacggacgaacctcatggcggagcattacgaagcgttagagagtaagattggggcaccttactatggcaaaaaggctgaaaaactaattagttgtgcagagtatctttcgtttaagagagacccggagacgggcaagttaaaactgtatcaagcccatttttgtaaagtgaggttatgtccgatgtgtgcgtggcgcaggtcgttaaaaattgcttatcacaataagttgatcgtagaggaagccaatagacagtacggctgcggatggatttttctcacgctgacgattcgaaatgtaaagggagaacggctgaagccacaaatttctgcgatgatggaaggctttaggaaactgttccagtacaaaaaagtaaaaacttcggttcttggatttttcagagctttagagattaccaaaaatcatgaagaagatacatatcatcctcattttcatgtgttgataccagtaaggaaaaattattttgggaaaaactatattaagcaggcggagtggacgagcctttggaaaaaggcgatgaaattggattacactccaattgtcgatattcgtcgagtgaaaggtaaagctaagattgacgctgaacagattgaaaacgatgtgcggaacgcaatgatggagcaaaaagctgttctcgaaatctctaaatatccggttaaggatacggatgttgtgcgcggtaataaggtgactgaagacaatctgaacacggtgctttacttggatgatgcgttggcagctcgaaggttaattggatacggtggcattttgaaggagatacataaagagctgaatcttggtgatgcggaggacggcgatctggtcaagattgaggaagaagatgacgaggttgcaaatggtgcatttgaggttatggcttattggcatcctggcattaaaaattacataatcaaataaaaaaagcagacctttagaaggcctgcttttttaactaacccatttgtattgtgttgaaatatgttttgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagcgattcacaaaaaataggcacacgaaaaacaagttaagggatgcagtttatgcatcccttaacttaaaatactaaaaatgcccatattttttcctccttataaaattagtataattatagcacgagatctaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccggatcc

SEQ ID NO: 16 - DNA, 1523 bp, synthetic construct
  misc_feature 1..1523: note = synthetic
  source 1..1523: mol_type = other DNA
ggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatgacgaactttggacaccatttacgacaattaagggaacggaaaaaactgaccgtcaatcaactggcgatgtattccggcgtcagttcggcaggcatttcgcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggcaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacggcgagcatcgccagcgccggaaccgtttgaatgacattagtgatggaaaagacccatttgctgattttacggtatctggcgatgaaaatgccggccgggatgccgacgacggcggcgaacaatacgccgtatgccgacattaaaaagtggcggtaaaattcccccagcacatagcc

SEQ ID NO: 17 - DNA, 1523 bp, synthetic construct; identical to SEQ ID NO: 16
  misc_feature 1..1523: note = synthetic
  source 1..1523: mol_type = other DNA

SEQ ID NO: 18 - DNA, 1922 bp, synthetic construct
  misc_feature 1..1922: note = synthetic
  source 1..1922: mol_type = other DNA
ggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatgacgaactttggacaccatttacgacaattaagggaacggaaaaaactgaccgtcaatcaactggcgatgtattccggcgtcagttcggcaggcatttcgcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagagccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacggcgagcatcgccagcgccggaaccgtttgaatgacattagtgatggaaaagacccatttgctgattttacggtatctggcgatgaaaatgccggccgggatgccgacgacggcggcgaacaatacgccgtatgccgacattaaaaagtggcggtaaaattcccccagcacatagcc

SEQ ID NO: 19 - DNA, 1309 bp, synthetic construct
  misc_feature 1..1309: note = synthetic
  source 1..1309: mol_type = other DNA
ctctaatagcttcgctttgattgttgtattatttattttgctgatcattgtgggctccgcttttgtaaagtattaattcattcgcgcacgaagccttcttccgaaaaactgcaggttcatatcgaaaggcgctccgcttttcatatgagcctgtttttttgtaatttaaaaagaatttatttccacaataacaaaaaatgtttataataggataggctctgagctgacggctgacggcgctcggaaataaaaactctcccacaaatgaaacttttatacaacattttcgtcttatactgttgttaaggcaattcaataacggaaaggaaaagatcaatgttaaataagctaagaatgttgtatgaaaaaacaacaatgaagtggctgctcaacacttcctattactttctcattctggtgctgctgtttttaatttacggattccatacagcaaatacaggttcatatatttataatgacttttaatggagaagaaaacaatgaaactgattgaaacaattaaaaaataccacaaatgtgcttgatggatccgtcttattcggaagagctgcttccggagctgagcttgtttatgttctgcggagaaacgcttcctgcgtcagtcgccagacagcttaaagaaagatttccaaaggctcgtgtttttaacacatacggcccgaccgaagcgaccgttgccgtcacttccatcgaagtaacagacgacgtattaaacaagtattcttctcttcccgtcggttctgaaaagcccgaaaccgaaatcgtaatcatcaatgaagacggaaaagccgtccaagacggggaaaaaggcgagatcataattaccggagcaagcgtcagcaaaggctatttaggcgaaaaagcgctgacggaaaaagcgtttttctcttacaacggctcccctgcttaccgcaccggtgacgcaggttacaaagaaaacggccagctcttcttcctcggaagactcgatttccaaatcaaactgcacggctaccggattgagcttgaagaaatcgaatatcaaatcaatcagtcccgatatgtgcagtcggcagtcgttattccattctaccgcgaggagaaaatcgagtatctgatcgccatgattgtgcctgccgaacacgatttcgaaaaggaataccagttaaccagcgcgattaagaaagatttgggcagcaagctgccggcctatatgattccgagaaaattcatgtatcaaaaagagattccgatgacagcaaacggtaaaatcgaccgcaagagattaaaagaagaggtaaccgtatga

SEQ ID NO: 20 - DNA, 2067 bp, synthetic construct
  misc_feature 1..2067: note = synthetic
  source 1..2067: mol_type = other DNA
gggtacctccatggtaaagtttcgacttaaagcggagatcaaaaaatagcctggtttaccattttccacatttttaaatttatacccctttcaaaagtcataaaactatcatattctgtaataccacttttcggaggtgagaatatgtcatactctaatagcttcgctttgattgttgtattatttattttgctgatcattgtgggctccgcttttgtaaagtattaattcattcgcgcacgaagccttcttccgaaaaactgcaggttcatatcgaaaggcgctccgcttttcatatgagcctgtttttttgtaatttaaaaagaatttatttccacaataacaaaaaatgtttataataggataggctctgagctgacggctgacggcgctcggaaataaaaactctcccacaaatgaaacttttatacaacattttcgtcttatactgttgttaaggcaattcaataacggaaaggaaaagatcaatgttaaataagctaagaatgttgtatgaaaaaacaacaatgaagtggctgctcaacacttcctattactttctcattctggtgctgctgtttttaatttacggattccatacagcaaatacaggttcatatatttataatgacttttaatggagaagaaaacaatgaaactgattgaaacaattaaaaaataccacaaatgtgcttgatggatccgtcttattcggaagagctgcttccggagctgagcttgtttatgttctgcggagaaacgcttcctgcgtcagtcgccagacagcttaaagaaagatttccaaaggctcgtgtttttaacacatacggcccgaccgaagcgaccgttgccgtcacttccatcgaagtaacagacgacgtattaaacaagtattcttctcttcccgtcggttctgaaaagcccgaaaccgaaatcgtaatcatcaatgaagacggaaaagccgtccaagacggggaaaaaggcgagatcataattaccggagcaagcgtcagcaaaggctatttaggcgaaaaagcgctgacggaaaaagcgtttttctcttacaacggctcccctgcttaccgcaccggtgacgcaggttacaaagaaaacggccagctcttcttcctcggaagactcgatttccaaatcaaactgcacggctaccggattgagcttgaagaaatcgaatatcaaatcaatcagtcccgatatgtgcagtcggcagtcgttattccattctaccgcgaggagaaaatcgagtatctgatcgccatgattgtgcctgccgaacacgatttcgaaaaggaataccagttaaccagcgcgattaagaaagatttgggcagcaagctgccggcctatatgattccgagaaaattcatgtatcaaaaagagattccgatgacagcaaacggtaaaatcgaccgcaagagattaaaagaagaggtaaccgtatgacgccttatggttcatttcttttcttcattatattaggaattttactggcgccgaccatcattctcggattgaacggaaaaagcttccgcttatataatatggcggtttccgttctggttctggcgcttattttttcgaacagcctgcacgggctgatcatgctgtgcctgtttacgctttggcagacggttctgatcaaaggctatatcgcttaccgtctaaaagcgaacagcggcatcgtattttgtctggctgcagcagcttctatcctgcctctggcactgtcaaagctgctgccgtttttcgccgttgacaactgggcaacatttctcggaatctcgtatttaacttttaaaggggttcagctcatcattgaaacccgcgacggtctcattaaaaagcagctcccaatcagcagactgctttactttattctcttttttccaaccatctcgtcaggtccgatcgacaggtaccgccgttttgaaaaagacgatcagacggtttggacaaaggagcaatacgaagaactgctttacaaaggaatcaataaaatttttctcggctttttgtacaaattcattatcggctactgcattaatacgt

SEQ ID NO: 21 - DNA, 2767 bp, synthetic construct
  misc_feature 1..2767: note = synthetic
  source 1..2767: mol_type = other DNA
gggtacctccatggtaaagtttcgacttaaagcggagatcaaaaaatagcctggtttaccattttccacatttttaaatttatacccctttcaaaagtcataaaactatcatattctgtaataccacttttcggaggtgagaatatgtcatactctaatagcttcgctttgattgttgtattatttattttgctgatcattgtgggctccgcttttgtaaagtattaattcattcgcgcacgaagccttcttccgaaaaactgcaggttcatatcgaaaggcgctccgcttttcatatgagcctgtttttttgtaatttaaaaagaatttatttccacaataacaaaaaatgtttataataggataggctctgagctgacggctgacggcgctcggaaataaaaactctcccacaaatgaaacttttatacaacattttcgtcttatactgttgttaaggcaattcaataacggaaaggaaaagatcaatgttaaataagctaagaatgttgtatgaaaaaacaacaatgaagtggctgctcaacacttcctattactttctcattctggtgctgctgtttttaatttacggattccatacagcaaatacaggttcatatatttataatgacttttaatggagaagaaaacaatgaaactgattgaaacaattaaaaaatacgcgcaaacacaacccgatacccttgcttttgtgaatgaagaagaaaagctgacatacggagagctttggtcacaatctgagcgccttgccgcaaggattcaaagcgaagctttaacagatgcttctccgatcattgtctacggacatatgaaaccggttatggcggtttccttcctggcttgtgtaaaagccgggcatccgtacattccagttgacgtatcgattccggcagaccggattctgaaaatcatcaacagttcaaaagccgagcttctgctcaataattcaggcacttcggttgacacgggagatgccctcatttccgtagtggaaccgggcgttcttgaaggagacgggatgccggagacagatcccggacgctgggtacatggggaagatactttttacattatctacacatccggaagcacgggaaatccaaaaggcgtgcaaatctcagccgataaccttcagagcttcacagattggatcacgaatgactttccggtggaaagcggccaagtgttcttaaatcaagccccgttttcctttgatttatcggtaatggacctttatccgtgcctgcagtccggaggcaccttatggacggttacgaaagacatgattaatcggccgaaactattgtttgaagcactgaaacagtccaacgtcaacgtctggacatcgacgccttcattcgcacaaatgtgcttgatggatccgtcttattcggaagagctgcttccggagctgagcttgtttatgttctgcggagaaacgcttcctgcgtcagtcgccagacagcttaaagaaagatttccaaaggctcgtgtttttaacacatacggcccgaccgaagcgaccgttgccgtcacttccatcgaagtaacagacgacgtattaaacaagtattcttctcttcccgtcggttctgaaaagcccgaaaccgaaatcgtaatcatcaatgaagacggaaaagccgtccaagacggggaaaaaggcgagatcataattaccggagcaagcgtcagcaaaggctatttaggcgaaaaagcgctgacggaaaaagcgtttttctcttacaacggctcccctgcttaccgcaccggtgacgcaggttacaaagaaaacggccagctcttcttcctcggaagactcgatttccaaatcaaactgcacggctaccggattgagcttgaagaaatcgaatatcaaatcaatcagtcccgatatgtgcagtcggcagtcgttattccattctaccgcgaggagaaaatcgagtatctgatcgccatgattgtgcctgccgaacacgatttcgaaaaggaataccagttaaccagcgcgattaagaaagatttgggcagcaagctgccggcctatatgattccgagaaaattcatgtatcaaaaagagattccgatgacagcaaacggtaaaatcgaccgcaagagattaaaagaagaggtaaccgtatgacgccttatggttcatttcttttcttcattatattaggaattttactggcgccgaccatcattctcggattgaacggaaaaagcttccgcttatataatatggcggtttccgttctggttctggcgcttattttttcgaacagcctgcacgggctgatcatgctgtgcctgtttacgctttggcagacggttctgatcaaaggctatatcgcttaccgtctaaaagcgaacagcggcatcgtattttgtctggctgcagcagcttctatcctgcctctggcactgtcaaagctgctgccgtttttcgccgttgacaactgggcaacatttctcggaatctcgtatttaacttttaaaggggttcagctcatcattgaaacccgcgacggtctcattaaaaagcagctcccaatcagcagactgctttactttattctcttttttccaaccatctcgtcaggtccgatcgacaggtaccgccgttttgaaaaagacgatcagacggtttggacaaaggagcaatacgaagaactgctttacaaaggaatcaataaaatttttctcggctttttgtacaaattcattatcggctactgcattaatacgt

SEQ ID NO: 22 - DNA, 3562 bp, synthetic construct
  misc_feature 1..3562: note = synthetic
  source 1..3562: mol_type = other DNA
cgcttcttgaaaacgaggtggaattttattttgagaaaaaacaaaccggtcttgaacagtctgtaaaaataaaaaaagaatgggtaaaagatattttaaaagaccgatatcaggatatgaaaaagaatcgtcttcaggccaaacctgatcaggagcctgttccgcttccgaagcaagcgaaaattaatcccgatgaaaaagtgattgccctcacatttgatgacggtccgaatcccgctacaacgaataaaatattaaacgctttacagaagcatgaagggcatgcgaccttctttgtgcttggaagcagagcccaatattatcccgaaacgataaaacggatgctgaaggaaggaaacgaagtcggcaaccattcctgggaccatccgttattgacaaggctgtcaaacgaaaaagcgtatcaggagattaacgacacgcaagaaatgatcgaaaaaatcagcggacacctgcctgtacacttgcgtcctccatacggcgggatcaatgattccgtccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgtccgcagatgctgctgaagagattattaaaaagctgaaagcaaaaggctatcaattggtaactgtatctcagcttgaagaagtgaagaagcagagaggctattgaataaatgagtagaaagcgccatatcggcatcttcaccccgatctatgtcagtataacctatatgacagccggaggtggagaggcggagaacggcacagcaagaagacaaagaagaagagagactgttgcctggacctccgaaacgcgctacaattcatttacaacacaggatggggtgagaatattgccggaatcagtgaagcaggcctcctaaaataaaaatctatattttaggaggtaaaacatgaattttcaaacaatcgagcttgacacatggtatagaaaatcttattttgaccattacatgaaggaagcgaaatgttctttcagcatcacggcaaacgtcaatgtgacaaatttgctcgccgtgctcaagaaaaagaagctcaagctgtatccggcttttatttatatcgtatcaagggtcattcattcgcgccctgagtttagaacaacgtttgatgacaaaggacagctgggttattgggaacaaatgcatccgtgctatgcgatttttcatcaggacgaccaaacgttttccgccctctggacggaatactcagacgatttttcgcagttttatcatcaatatcttctggacgccgagcgctttggagacaaaaggggcctttgggctaagccggacatcccgcccaatacgttttcagtttcttctattccatgggtgcgcttttcaaacttcaatttaaaccttgataacagcgaacacttgctgccgattattacaaacgggaaatacttttcagaaggcagggaaacatttttgcccgtttccttgcaagttcaccatgcagtgtgtgacggctatcatgccggcgcttttataaacgagttggaacggcttgccgccgattgtgaggagtggcttgtgtgacagaggaaaggccgatatgattcggccttttttatatgtacttcttagcgggtctctaaaataaccaaaaagcaaggactgctgaaagggctgacataagccttttgccggcggtccttttttaattctgatttttcaaacttagccccctcgagaaaaccgcttgagtatcctccgctcatgttattgacctccttttcttgatcaatatatttgtatgagcggaaagaaaatttgactagacggatgcccctctgttgaaaatctttttacagcatgtcagaatatgatatgatacaattcaaaggaaagtttaaactgttatgattaggagtgtttgcatttatgaagaagattgcaattgcggcgattacagcgacaagcgtgctggctctcagcgcatgcagcgggggagattctgaggttgttgcggaaacaaaagctggaaatattacaaaagaagacctttatcaaacattaaaagacaatgccggagcggacgcactgaacatgcttgttcagcaaaaagtactcgatgataaatacgatgtctccgacaaagaaatcgacaaaaagctgaacgagtacaaaaaatcaatgggtgaccagctcaaccagctcattgaccaaaaaggcgaagacttcgtcaaagaacagatcaaatacgaacttctgatgcaaaaagccgcaaaggataacataaaagtaaccgatgatgacgtaaaagaatattatgacggcctgaaaggcaaaatccacttaagccacattcttgtgaaagaaaagaaaacggctgaagaagttgagaaaaagctgaaaaaaggcgaaaaattcgaagaccttgcaaaagagtattcaactgacggtacagccgaaaaaggcggcgacctcggctgggtcggcaaagacgataacatggacaaggatttcgtcaaagcggcatttgctttgaaaaccggcgaaatcagcggacctgtgaaatcccaattcggctatcacatcattaaaaaagacgaagaacgcggcaaatatgaagacatgaaaaaagagcttaaaaaagaagtccaagaacaaaagcaaaatgatcaaactgaactgcaatccgtcattgacaaacttgtcaaagatgctgatttaaaagtaaaagacaaagagttgaaaaaacaagtcgaccagcgtcaagctcagacaagcagcagcagctgaaagagcagagaggacggatttcctgaaggaaatccgtttttttattttgcccgtcttataaatttctttgattacattttataattaattttaacaaagtgtcatcagccctcaggaaggacttgctgacagtttgaatcgcataggtaaggcggggatgaaatggcaacgttatctgatgtagcaaagaaagcaaatgtgtcgaaaatgacggtatcgcgggtgatcaatcatcctgagactgtgacggatgaattgaaaaagcttgttcattccgcaatgaaggagctcaattatataccgaactatgcagcaagagcgctcgttcaaaacagaacacaggtcgtcaagctgctcatactggaagaaatggatacaacagaaccttattatatgaatctgttaacgggaatcagccgcgagctggaccgtcatcattatgctttgcagcttgtcacaaggaaatctctcaatatcggccagtgcgacggcattattgcgacggggttgagaaaagccgattttgaagggctcatcaaggtttttgaaaagcctgtcgttgtattcgggcaaaatgaaatgggctacgattttattgatgttaacaatgaaaaaggaacctatatggcaacacgtcacgtcattggtctgggcgtccgcaatgtcgtcttttttgggatcgatttggatgagc

SEQ ID NO: 23 - DNA, 794 bp, synthetic construct
  misc_feature 1..794: note = synthetic
  source 1..794: mol_type = other DNA
gcttcttgaaaacgaggtggaattttattttgagaaaaaacaaaccggtcttgaacagtctgtaaaaataaaaaaagaatgggtaaaagatattttaaaagaccgatatcaggatatgaaaaagaatcgtcttcaggccaaacctgatcaggagcctgttccgcttccgaagcaagcgaaaattaatcccgatgaaaaagtgattgccctcacatttgatgacggtccgaatcccgctacaacgaataaaatattaaacgctttacagaagcatgaagggcatgcgaccttctttgtgcttggaagcagagcccaatattatcccgaaacgataaaacggatgctgaaggaaggaaacgaagtcggcaaccattcctgggaccatccgttattgacaaggctgtcaaacgaaaaagcgtatcaggagattaacgacacgcaagaaatgatcgaaaaaatcagcggacacctgcctgtacacttgcgtcctccatacggcgggatcaatgattccgtccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgtccgcagatgctgctgaagagattattaaaaagctgaaagcaaaaggctatcaattggtaactgtatctcagcttgaagaagtgaagaagcagagaggctattgaataaatgagtagaaagcgccatatcggc

SEQ ID NO: 24 - DNA, 216 bp, synthetic construct
  misc_feature 1..216: note = synthetic
  source 1..216: mol_type = other DNA
atcttcaccccgatctatgtcagtataacctatatgacagccggaggtggagaggcggagaacggcacagcaagaagacaaagaagaagagagactgttgcctggacctccgaaacgcgctacaattcatttacaacacaggatggggtgagaatattgccggaatcagtgaagcaggcctcctaaaataaaaatctatattttaggaggtaaaac

SEQ ID NO: 25 - DNA, 648 bp, synthetic construct
  misc_feature 1..648: note = synthetic
  source 1..648: mol_type = other DNA
atgaattttcaaacaatcgagcttgacacatggtatagaaaatcttattttgaccattacatgaaggaagcgaaatgttctttcagcatcacggcaaacgtcaatgtgacaaatttgctcgccgtgctcaagaaaaagaagctcaagctgtatccggcttttatttatatcgtatcaagggtcattcattcgcgccctgagtttagaacaacgtttgatgacaaaggacagctgggttattgggaacaaatgcatccgtgctatgcgatttttcatcaggacgaccaaacgttttccgccctctggacggaatactcagacgatttttcgcagttttatcatcaatatcttctggacgccgagcgctttggagacaaaaggggcctttgggctaagccggacatcccgcccaatacgttttcagtttcttctattccatgggtgcgcttttcaaacttcaatttaaaccttgataacagcgaacacttgctgccgattattacaaacgggaaatacttttcagaaggcagggaaacatttttgcccgtttccttgcaagttcaccatgcagtgtgtgacggctatcatgccggcgcttttataaacgagttggaacggcttgccgccgattgtgaggagtggcttgtg

SEQ ID NO: 26 - DNA, 148 bp, synthetic construct
  misc_feature 1..148: note = synthetic
  source 1..148: mol_type = other DNA
tgacagaggaaaggccgatatgattcggccttttttatatgtacttcttagcgggtctctaaaataaccaaaaagcaaggactgctgaaagggctgacataagccttttgccggcggtccttttttaattctgatttttcaaacttag

SEQ ID NO: 27 - DNA, 60 bp, synthetic construct
  misc_feature 1..60: note = synthetic
  source 1..60: mol_type = other DNA
tgacagaggaaaggccgatatgattcggccttttttatatgtacttcttagcgggtctct

SEQ ID NO: 28 - DNA, 98 bp, synthetic construct
  misc_feature 1..98: note = synthetic
  source 1..98: mol_type = other DNA
aaaataaccaaaaagcaaggactgctgaaagggctgacataagccttttgccggcggtccttttttaattctgatttttcaaacttagccccctcgag

SEQ ID NO: 29 - DNA, 190 bp, synthetic construct
  misc_feature 1..190: note = synthetic
  source 1..190: mol_type = other DNA
aaaaccgcttgagtatcctccgctcatgttattgacctccttttcttgatcaatatatttgtatgagcggaaagaaaatttgactagacggatgcccctctgttgaaaatctttttacagcatgtcagaatatgatatgatacaattcaaaggaaagtttaaactgttatgattaggagtgtttgcattt

SEQ ID NO: 30 - DNA, 861 bp, synthetic construct
  misc_feature 1..861: note = synthetic
  source 1..861: mol_type = other DNA
atgaagaagattgcaattgcggcgattacagcgacaagcgtgctggctctcagcgcatgcagcgggggagattctgaggttgttgcggaaacaaaagctggaaatattacaaaagaagacctttatcaaacattaaaagacaatgccggagcggacgcactgaacatgcttgttcagcaaaaagtactcgatgataaatacgatgtctccgacaaagaaatcgacaaaaagctgaacgagtacaaaaaatcaatgggtgaccagctcaaccagctcattgaccaaaaaggcgaagacttcgtcaaagaacagatcaaatacgaacttctgatgcaaaaagccgcaaaggataacataaaagtaaccgatgatgacgtaaaagaatattatgacggcctgaaaggcaaaatccacttaagccacattcttgtgaaagaaaagaaaacggctgaagaagttgagaaaaagctgaaaaaaggcgaaaaattcgaagaccttgcaaaagagtattcaactgacggtacagccgaaaaaggcggcgacctcggctgggtcggcaaagacgataacatggacaaggatttcgtcaaagcggcatttgctttgaaaaccggcgaaatcagcggacctgtgaaatcccaattcggctatcacatcattaaaaaagacgaagaacgcggcaaatatgaagacatgaaaaaagagcttaaaaaagaagtccaagaacaaaagcaaaatgatcaaactgaactgcaatccgtcattgacaaacttgtcaaagatgctgatttaaaagtaaaagacaaagagttgaaaaaacaagtcgaccagcgtcaagctcagacaagcagcagcagctga

SEQ ID NO: 31 - DNA, 34 bp, synthetic construct
  misc_feature 1..34: note = synthetic
  source 1..34: mol_type = other DNA
cggatttcctgaaggaaatccgtttttttatttt

SEQ ID NO: 32 - DNA, 646 bp, synthetic construct
  misc_feature 1..646: note = synthetic
  source 1..646: mol_type = other DNA
gcccgtcttataaatttctttgattacattttataattaattttaacaaagtgtcatcagccctcaggaaggacttgctgacagtttgaatcgcataggtaaggcggggatgaaatggcaacgttatctgatgtagcaaagaaagcaaatgtgtcgaaaatgacggtatcgcgggtgatcaatcatcctgagactgtgacggatgaattgaaaaagcttgttcattccgcaatgaaggagctcaattatataccgaactatgcagcaagagcgctcgttcaaaacagaacacaggtcgtcaagctgctcatactggaagaaatggatacaacagaaccttattatatgaatctgttaacgggaatcagccgcgagctggaccgtcatcattatgctttgcagcttgtcacaaggaaatctctcaatatcggccagtgcgacggcattattgcgacggggttgagaaaagccgattttgaagggctcatcaaggtttttgaaaagcctgtcgttgtattcgggcaaaatgaaatgggctacgattttattgatgttaacaatgaaaaaggaacctatatggcaacacgtcacgtcattggtctgggcgtccgcaatgtcgtcttttttgggatcgatttggatgagc

SEQ ID NO: 33 - DNA, 3562 bp, synthetic construct; identical to SEQ ID NO: 22
  misc_feature 1..3562: note = synthetic
  source 1..3562: mol_type = other DNA

SEQ ID NO: 34 - DNA, 2698 bp, synthetic construct
  misc_feature 1..2698: note = synthetic
  source 1..2698: mol_type = other DNA
cgcttcttgaaaacgaggtggaattttattttgagaaaaaacaaaccggtcttgaacagtctgtaaaaataaaaaaagaatgggtaaaagatattttaaaagaccgatatcaggatatgaaaaagaatcgtcttcaggccaaacctgatcaggagcctgttccgcttccgaagcaagcgaaaattaatcccgatgaaaaagtgattgccctcacatttgatgacggtccgaatcccgctacaacgaataaaatattaaacgctttacagaagcatgaagggcatgcgaccttctttgtgcttggaagcagagcccaatattatcccgaaacgataaaacggatgctgaaggaaggaaacgaagtcggcaaccattcctgggaccatccgttattgacaaggctgtcaaacgaaaaagcgtatcaggagattaacgacacgcaagaaatgatcgaaaaaatcagcggacacctgcctgtacacttgcgtcctccatacggcgggatcaatgattccgtccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgtccgcagatgctgctgaagagattattaaaaagctgaaagcaaaaggctatcaattggtaactgtatctcagcttgaagaagtgaagaagcagagaggctattgaataaatgagtagaaagcgccatatcggctgacagaggaaaggccgatatgattcggccttttttatatgtacttcttagcgggtctctaaaataaccaaaaagcaaggactgctgaaagggctgacataagccttttgccggcggtccttttttaattctgatttttcaaacttagccccctcgagaaaaccgcttgagtatcctccgctcatgttattgacctccttttcttgatcaatatatttgtatgagcggaaagaaaatttgactagacggatgcccctctgttgaaaatctttttacagcatgtcagaatatgatatgatacaattcaaaggaaagtttaaactgttatgattaggagtgtttgcatttatgaagaagattgcaattgcggcgattacagcgacaagcgtgctggctctcagcgcatgcagcgggggagattctgaggttgttgcggaaacaaaagctggaaatattacaaaagaagacctttatcaaacattaaaagacaatgccggagcggacgcactgaacatgcttgttcagcaaaaagtactcgatgataaatacgatgtctccgacaaagaaatcgacaaaaagctgaacgagtacaaaaaatcaatgggtgaccagctcaaccagctcattgaccaaaaaggcgaagacttcgtcaaagaacagatcaaatacgaacttctgatgcaaaaagccgcaaaggataacataaaagtaaccgatgatgacgtaaaagaatattatgacggcctgaaaggcaaaatccacttaagccacattcttgtgaaagaaaagaaaacggctgaagaagttgagaaaaagctgaaaaaaggcgaaaaattcgaagaccttgcaaaagagtattcaactgacggtacagccgaaaaaggcggcgacctcggctgggtcggcaaagacgataacatggacaaggatttcgtcaaagcggcatttgctttgaaaaccggcgaaatcagcggacctgtgaaatcccaattcggctatcacatcattaaaaaagacgaagaacgcggcaaatatgaagacatgaaaaaagagcttaaaaaagaagtccaagaacaaaagcaaaatgatcaaactgaactgcaatccgtcattgacaaacttgtcaaagatgctgatttaaaagtaaaagacaaagagttgaaaaaacaagtcgaccagcgtcaagctcagacaagcagcagcagctgaaagagcagagaggacggatttcctgaaggaaatccgtttttttattttgcccgtcttataaatttctttgattacattttataattaattttaacaaagtgtcatcagccctcaggaaggacttgctgacagtttgaatcgcataggtaaggcggggatgaaatggcaacgttatctgatgtagcaaagaaagcaaatgtgtcgaaaatgacggtatcgcgggtgatcaatcatcctgagactgtgacggatgaattgaaaaagcttgttcattccgcaatgaaggagctcaattatataccgaactatgcagcaagagcgctcgttcaaaacagaacacaggtcgtcaagctgctcatactggaagaaatggatacaacagaaccttattatatgaatctgttaacgggaatcagccgcgagctggaccgtcatcattatgctttgcagcttgtcacaaggaaatctctcaatatcggccagtgcgacggcattattgcgacggggttgagaaaagccgattttgaagggctcatcaaggtttttgaaaagcctgtcgttgtattcgggcaaaatgaaatgggctacgattttattgatgttaacaatgaaaaaggaacctatatggcaacacgtcacgtcattggtctgggcgtccgcaatgtcgtcttttttgggatcgatttggatgagc

SEQ ID NO: 35 - DNA, 3562 bp, synthetic construct; identical to SEQ ID NO: 22
  misc_feature 1..3562: note = synthetic
  source 1..3562: mol_type = other DNA

SEQ ID NO: 36 - DNA, 1455 bp, synthetic construct
  misc_feature 1..1455: note = synthetic
  source 1..1455: mol_type = other DNA
caccacaacggaacaaacggcacaatgatgcagtattttgaatggcacctgccgaatgatggacagcattggaatagactgagaaacgacgcagcgaacctgaagaaccttggcatcaacgcagtctggattccgcctgcgtggaaaggcacatcacaaaatgatgtcggctatggcgcatatgacctgtacgacctgggagagttcaaccagaagggaacaatcagaacgaaatatggaacaagatcacaactgcaaagcgctatcgcgcgcctgcaaaataatggcatccaagtttttggcgacgtggtcatgaaccacaaaggcggagcagacggaacggaacgcgttcaagcggtcgaggtgaatccgagcaacagaaaccaagaggttacgggcgaatacacgatcgaagcctggacaaagttcgactttccgggcagaggcaatacacactctagcttcaagtggagatggtatcactttgacggcacggactgggatcaaagcagaaacctgaataacagaatctataagtttacaggcaaagcatgggattgggaggtggacacagaaaacggaaactatgactatctgatgtatgctgacgtcgacatggatcatccggaggtcatcaatgagctgagaagatggggcgtttggtacacgaacacactgaacctggatggattcagaattgacgcagtcaaacacatcaagtaccagtttacaagagactggcttaaccacgtgagatcaacaacgggaaagaacaatatgttcgccgttgcggagttttggaagaatgatctgggcgcaatcgagaactatcttagcaagacgaactggaatcatagcgtcttcgatgtcccgctgcactacaatctgtataatgcatcaaagtcaggcggcaactacgatatgagacaaatcctgaatggaacggtcgtctcaaaacacccgatccacgccgtcacgtttgtcgataatcacgattcacaaccggcagaagcccttgagagctttgttgaggcatggttcaaaccgctggcctatgctctgatcctgacaagagagcaaggctatccgtcagtgttttacggcgattactacggcattccgacacatggcgtcgcagccatgaaaggaaagattgatccgattctggaggctagacagaaatacgcctacggaacgcaacacgattaccttgatcaccataacatcatcggatggacgagagagggaaactcagcacatcctaactcaggccttgcaacaattatgtcagatggaccgggaggcagcaaatggatgtacgttggcagacacaaggcaggacaggtttggagagatatcacgggcaatagaacaggaacagttacaatcaacgctgacggctggggcaattttagcgttaatggcggctcagttagcatctgggtgaataaatga

SEQ ID NO: 37 - DNA, 95 bp, synthetic construct
  misc_feature 1..95: note = synthetic
  source 1..95: mol_type = other DNA
gtcgctgataaacagctgacatcaatatcctattttttcaaaaaatattttaaaaagttgttgacttaaaagaagctaaatgttatagtaataaa

SEQ ID NO: 38 - DNA, 57 bp, synthetic construct
  misc_feature 1..57: note = synthetic
  source 1..57: mol_type = other DNA
acagaatagtcttttaagtaagtctactctgaatttttttaaaaggagagggtaaag

SEQ ID NO: 39 - DNA, 87 bp, synthetic construct
  misc_feature 1..87: note = synthetic
  source 1..87: mol_type = other DNA
atgaaacaacaaaaacggctttacgcccgattgctgacgctgttatttgcgctcatcttcttgctgcctcattctgcagctagcgca

SEQ ID NO: 40 - DNA, 34 bp, synthetic construct; identical to SEQ ID NO: 31
  misc_feature 1..34: note = synthetic
  source 1..34: mol_type = other DNA

SEQ ID NO: 41 - DNA, 91 bp, synthetic construct
  misc_feature 1..91: note = synthetic
  source 1..91: mol_type = other DNA
gctgataaacagctgacatcaactaaaagtttcattaaatactttgaaaaaagttgttgacttaaaagaagctaaatgttatagtaattgt

SEQ ID NO: 42 - DNA, 1290 bp, synthetic construct
  misc_feature 1..1290: note = synthetic
  source 1..1290: mol_type = other DNA
gatcaagcaggaaaaagcccggcaggcgtcagatatcatggcggtgatgaaatcatccttcagggctttcattggaacgtcgtcagagaagtcccgtatcagtggtataacatcctgagacaaaaagcgatcacaattgccgctgatggcttttccgcaatctggatgccgcctccgtggagagattttagcagctggacgtcaggagataaaagcggaggcggcgaaggatatttttggcatgactttaacaaaaacggcctgtatggaagcgatgctcaactgagacaagcagcaggaaagcttggaggagcaggagtcaaagtcctgtacgatgtcgtcccgaaccatatgaaccgcttttatccggacaaagaaatcaatctgccggcaggccaaagattttggagaaacgattgcccggacccgggaaatggaccgaatgattgcgatgatggcgatccttttctgggcggcgaagcggatctgaatacaggccatccgcaaatctatcagatgtttcgggacgaatttcgcaatctgagaagcggatatggagcgggcggatttcgctttgattttgtcagaggctatgccggcgaaagagttgatctgtggatgagcgattcagcggatgaaagcttttgcgtcggcgaactttggaaatggccgagcgaatatccgccgagcgattggagaaatagagcgagctggcaggaaatcatcaaagattggagcgatagatcaaaatgcccggtctttgactttgccctgaaagaacgcatgcaaaatggaagcgtcgccgattggaaacatggcctgaacggaaatccggacccgagatggagagaagtcgccgtcacgtttgtcgataaccatgacacaggatatagcccgggacaaaatggaggacaacataaatggccgcttccggatgcgcttatcagacaggcgtatgcctatatccttacatcaccgggaacaccggttgtttattggccgcatatgtatgattggggctatggcgatttcatccgccaactgatccaggttagaagaacagcaggagtcagagcggatagcgccattagctttcatacgggctatagcggacttgtcgctacagttcagggcagccaacaaacactggtcgtcgccctgaatagcgatctggacaatccgaaacaagttgctagcggcagctttagcctggcagtcaatgccgaaaatggccaagtcagagtctggagaagcggaagcggagatggaggaggaaatgacggcggctaa

SEQ ID NO: 43 - protein, 286 aa, Bacillus licheniformis
MKKIAIAAITATSVLALSACSGGDSEVVAETKAGNITKEDLYQTLKDNAGADALNMLVQQKVLDDKYDVSDKEIDKKLNEYKKSMGDQLNQLIDQKGEDFVKEQIKYELLMQKAAKDNIKVTDDDVKEYYDGLKGKIHLSHILVKEKKTAEEVEKKLKKGEKFEDLAKEYSTDGTAEKGGDLGWVGKDDNMDKDFVKAAFALKTGEISGPVKSQFGYHIIKKDEERGKYEDMKKELKKEVQEQKQNDQTELQSVIDKLVKDADLKVKDKELKKQVDQRQAQTSSSS